SEQUENCE LISTING (208 sequences)

SEQ ID NO: 1 - protein, 8 aa, synthetic construct
GFTFSNAW

SEQ ID NO: 2 - protein, 10 aa, synthetic construct
IKSKTDGGTT

SEQ ID NO: 3 - protein, 16 aa, synthetic construct
TTGWELQDYYYYGMDV

SEQ ID NO: 4 - protein, 125 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTGWELQDYYYYGMDVWGQGTTVTVSS

SEQ ID NO: 5 - DNA, 24 bp, synthetic construct
  source 1..24: mol_type = other DNA
ggattcactttcagtaacgcctgg

SEQ ID NO: 6 - DNA, 30 bp, synthetic construct
  source 1..30: mol_type = other DNA
attaaaagcaaaactgatggtgggacaaca

SEQ ID NO: 7 - DNA, 48 bp, synthetic construct
  source 1..48: mol_type = other DNA
accacagggtgggagctacaggactactactactacggtatggacgtc

SEQ ID NO: 8 - DNA, 375 bp, synthetic construct
  source 1..375: mol_type = other DNA
gaggtgcagctggtggagtcggggggaggcttggtaaagcctggggggtcccttagactctcctgtgcagcctctggattcactttcagtaacgcctggatgagctgggtccgccaggctccagggaaggggctggagtgggttggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagggtgggagctacaggactactactactacggtatggacgtctggggccaagggaccacggtcaccgtctcctca

SEQ ID NO: 9 - protein, 6 aa, synthetic construct
QSVSSN

SEQ ID NO: 10 - skipped

SEQ ID NO: 11 - protein, 9 aa, synthetic construct
QQYNNWPLT

SEQ ID NO: 12 - protein, 106 aa, synthetic construct
IVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 13 - DNA, 18 bp, synthetic construct
  source 1..18: mol_type = other DNA
cagagtgttagcagcaac

SEQ ID NO: 14 - skipped

SEQ ID NO: 15 - DNA, 27 bp, synthetic construct
  source 1..27: mol_type = other DNA
cagcagtataataactggccgctcact

SEQ ID NO: 16 - DNA, 318 bp, synthetic construct
  source 1..318: mol_type = other DNA
atagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgctcactttcggcggagggaccaaggtggagatcaaa

SEQ ID NO: 17 - protein, 8 aa, synthetic construct
GGSFSGYY

SEQ ID NO: 18 - protein, 7 aa, synthetic construct
INHSGST

SEQ ID NO: 19 - protein, 17 aa, synthetic construct
ARGDCSGGSCPYWYFDL

SEQ ID NO: 20 - protein, 123 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGDCSGGSCPYWYFDLWGRGTLVTVSS

SEQ ID NO: 21 - DNA, 25 bp, synthetic construct
  source 1..25: mol_type = other DNA
ggtgggtccttcagtggttactact

SEQ ID NO: 22 - DNA, 21 bp, synthetic construct
  source 1..21: mol_type = other DNA
atcaatcatagtggaagcacc

SEQ ID NO: 23 - DNA, 51 bp, synthetic construct
  source 1..51: mol_type = other DNA
gcgagaggtgattgtagtggtggtagctgtccttactggtacttcgatctc

SEQ ID NO: 24 - DNA, 369 bp, synthetic construct
  source 1..369: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaggtgattgtagtggtggtagctgtccttactggtacttcgatctctggggccgtggcaccctggtcactgtctcctca

SEQ ID NO: 25 - protein, 11 aa, synthetic construct
QSLLHSNGYNY

SEQ ID NO: 26 - skipped

SEQ ID NO: 27 - protein, 9 aa, synthetic construct
MQALQTPRT

SEQ ID NO: 28 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKVEIK

SEQ ID NO: 29 - DNA, 33 bp, synthetic construct
  source 1..33: mol_type = other DNA
cagagcctcctgcatagtaatggatacaactat

SEQ ID NO: 30 - skipped

SEQ ID NO: 31 - DNA, 27 bp, synthetic construct
  source 1..27: mol_type = other DNA
atgcaagctctacaaactcctcggacg

SEQ ID NO: 32 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcggacgttcggccaagggaccaaggtggaaatcaaa

SEQ ID NO: 33 - protein, 8 aa, synthetic construct
GFTFRNYA

SEQ ID NO: 34 - protein, 8 aa, synthetic construct
IRGSGSRT

SEQ ID NO: 35 - protein, 17 aa, synthetic construct
AKDEGFGDLLAHYVMDA

SEQ ID NO: 36 - protein, 124 aa, synthetic construct
EVQLLESGGGLVQPGGSLRLSCEASGFTFRNYAMSWVRQAPGKGLEWVSAIRGSGSRTYYADSVKGRFTISRDNSKNTLYLQMNSLRAEDTAVYYCAKDEGFGDLLAHYVMDAWGQGASVTVSS

SEQ ID NO: 37 - DNA, 24 bp, synthetic construct
  source 1..24: mol_type = other DNA
ggattcacctttagaaactatgcc

SEQ ID NO: 38 - DNA, 24 bp, synthetic construct
  source 1..24: mol_type = other DNA
attcgtggtagtggtagtagaaca

SEQ ID NO: 39 - DNA, 51 bp, synthetic construct
  source 1..51: mol_type = other DNA
gcgaaagatgaggggttcggggacttattagcgcactatgttatggatgcc

SEQ ID NO: 40 - DNA, 372 bp, synthetic construct
  source 1..372: mol_type = other DNA
gaggtgcagctgttggagtctgggggaggcttggtacagcctggggggtccctgagactctcctgtgaagcctcaggattcacctttagaaactatgccatgagctgggtccgccaggctccagggaaggggctggagtgggtctcagctattcgtggtagtggtagtagaacatactacgcagactctgtgaagggccggttcaccatctccagagacaattccaagaacacgctgtatctgcagatgaacagcctgagagccgaggacacggccgtgtattactgcgcgaaagatgaggggttcggggacttattagcgcactatgttatggatgcctggggtcaaggagcttcagtcactgtctcctca

SEQ ID NO: 41 - protein, 6 aa, synthetic construct
QSVSSN

SEQ ID NO: 42 - skipped

SEQ ID NO: 43 - protein, 9 aa, synthetic construct
QQYNNWPIT

SEQ ID NO: 44 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPITFGQGTRLEIK

SEQ ID NO: 45 - DNA, 18 bp, synthetic construct
  source 1..18: mol_type = other DNA
cagagtgttagcagcaac

SEQ ID NO: 46 - skipped

SEQ ID NO: 47 - DNA, 27 bp, synthetic construct
  source 1..27: mol_type = other DNA
cagcagtataataactggccgatcacc

SEQ ID NO: 48 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgatcaccttcggccaagggacacgactggagattaaa

SEQ ID NO: 49 - protein, 8 aa, synthetic construct
GFTFSNYA

SEQ ID NO: 50 - protein, 8 aa, synthetic construct
ISMSGGST

SEQ ID NO: 51 - protein, 14 aa, synthetic construct
AKDEYSSGWYQFDY

SEQ ID NO: 52 - protein, 121 aa, synthetic construct
EVQLLESGGGLVQPGGSLRLSCAASGFTFSNYAMNWVRQAPGKGLEWVSAISMSGGSTYYADSVKGRFTISRDNSKNTLYLQMNTLRAEDTAVYYCAKDEYSSGWYQFDYWGQGTLVTVSS

SEQ ID NO: 53 - DNA, 24 bp, synthetic construct
  source 1..24: mol_type = other DNA
ggattcacctttagcaactatgcc

SEQ ID NO: 54 - DNA, 24 bp, synthetic construct
  source 1..24: mol_type = other DNA
attagtatgagtggtggtagcaca

SEQ ID NO: 55 - DNA, 42 bp, synthetic construct
  source 1..42: mol_type = other DNA
gcgaaagacgaatatagcagtggctggtaccaatttgactat

SEQ ID NO: 56 - DNA, 363 bp, synthetic construct
  source 1..363: mol_type = other DNA
gaggtgcagttgttggagtctggggggggcttggtacagcctggggggtccctgagactctcctgtgcagcctctggattcacctttagcaactatgccatgaactgggtccgccaggctccagggaaggggctggagtgggtctcagctattagtatgagtggtggtagcacatactatgcagactccgtgaagggccggttcaccatctccagagacaattccaagaacacgctgtatctgcaaatgaacaccctgagagccgaggacacggccgtatattactgtgcgaaagacgaatatagcagtggctggtaccaatttgactattggggccagggaaccctggtcaccgtctcctca

SEQ ID NO: 57 - protein, 6 aa, synthetic construct
QSISSY

SEQ ID NO: 58 - skipped

SEQ ID NO: 59 - protein, 9 aa, synthetic construct
QQSYSTPLT

SEQ ID NO: 60 - protein, 107 aa, synthetic construct [sequence omitted]

SEQ ID NO: 61 - DNA, 18 bp, synthetic construct
  source 1..18: mol_type = other DNA
cagagcattagcagctat

SEQ ID NO: 62 - skipped

SEQ ID NO: 63 - DNA, 27 bp, synthetic construct
  source 1..27: mol_type = other DNA
caacagagttacagtaccccgctcact

SEQ ID NO: 64 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgctcactttcggcggagggaccaaggtggagatcaaa

SEQ ID NO: 65 - protein, 8 aa, synthetic construct
GFTFSTYG

SEQ ID NO: 66 - protein, 8 aa, synthetic construct
TWYDEYNK

SEQ ID NO: 67 - protein, 14 aa, synthetic construct
ARNVLRYFDWTLDY

SEQ ID NO: 68 - protein, 121 aa, synthetic construct
QVQLVESGGGVVQPGRSLRLSCAASGFTFSTYGMHWVRQAPGKGLEWVAVTWYDEYNKYYADSVKGRFTISRDNSKNMLYLQMNSLRAEDTAMYYCARNVLRYFDWTLDYWGQGTLVTVSS

SEQ ID NO: 69 - DNA, 24 bp, synthetic construct
  source 1..24: mol_type = other DNA
ggattcaccttcagtacctatggc

SEQ ID NO: 70 - DNA, 24 bp, synthetic construct
  source 1..24: mol_type = other DNA
acatggtatgatgaatataataaa

SEQ ID NO: 71 - DNA, 42 bp, synthetic construct
  source 1..42: mol_type = other DNA
gcgaggaacgtattacgatattttgactggacccttgactac

SEQ ID NO: 72 - DNA, 363 bp, synthetic construct
  source 1..363: mol_type = other DNA
caggtgcagctggtggagtctgggggaggcgtggtccagcctgggaggtccctgagactctcctgtgcagcgtctggattcaccttcagtacctatggcatgcactgggtccgccaggctccaggcaaggggctggagtgggtggcagttacatggtatgatgaatataataaatactatgcagactccgtgaagggccgcttcaccatctccagagacaattccaagaacatgttgtatctgcaaatgaacagcctgagagccgaggacacggctatgtattactgtgcgaggaacgtattacgatattttgactggacccttgactactggggccagggaaccctggtcaccgtctcctcg

SEQ ID NO: 73 - protein, 6 aa, synthetic construct
QSVSSY

SEQ ID NO: 74 - skipped

SEQ ID NO: 75 - protein, 9 aa, synthetic construct
QQRSNWPPT

SEQ ID NO: 76 - protein, 106 aa, synthetic construct
IVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 77 - DNA, 18 bp, synthetic construct
  source 1..18: mol_type = other DNA
cagagtgttagcagctac

SEQ ID NO: 78 - skipped

SEQ ID NO: 79 - DNA, 27 bp, synthetic construct
  source 1..27: mol_type = other DNA
cagcagcgtagcaactggcctcccact

SEQ ID NO: 80 - DNA, 318 bp, synthetic construct
  source 1..318: mol_type = other DNA
attgtgttgacacagtctccagccaccctgtctttgtctccaggggaaagagccaccctctcctgcagggccagtcagagtgttagcagctacttagcctggtatcaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctcccacttttggccaggggaccaaggtggagatcaaa

SEQ ID NO: 81 - protein, 8 aa, synthetic construct
GFTFSSYA

SEQ ID NO: 82 - protein, 8 aa, synthetic construct
ISGSGGST

SEQ ID NO: 83 - protein, 15 aa, synthetic construct
AKAGIAVAGGWYFDL

SEQ ID NO: 84 - protein, 122 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKAGIAVAGGWYFDLWGRGTLVTVSS

SEQ ID NO: 85 - DNA, 24 bp, synthetic construct
  source 1..24: mol_type = other DNA
ggattcacctttagcagctatgcc

SEQ ID NO: 86 - DNA, 24 bp, synthetic construct
  source 1..24: mol_type = other DNA
attagtggtagtggtggtagcaca

SEQ ID NO: 87 - DNA, 45 bp, synthetic construct
  source 1..45: mol_type = other DNA
gcgaaagccggtatagcagtggctgggggatggtacttcgatctc

SEQ ID NO: 88 - DNA, 366 bp, synthetic construct
  source 1..366: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagccggtatagcagtggctgggggatggtacttcgatctctggggccgtggcaccctggtcactgtctcctca

SEQ ID NO: 89 - protein, 6 aa, synthetic construct
QSVSSY

SEQ ID NO: 90 - skipped

SEQ ID NO: 91 - protein, 9 aa, synthetic construct
QQRSNWPPT

SEQ ID NO: 92 - protein, 106 aa, synthetic construct
IVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKLEIK

SEQ ID NO: 93 - DNA, 18 bp, synthetic construct
  source 1..18: mol_type = other DNA
cagagtgttagcagctac

SEQ ID NO: 94 - skipped

SEQ ID NO: 95 - DNA, 27 bp, synthetic construct
  source 1..27: mol_type = other DNA
cagcagcgtagcaactggcctcccact

SEQ ID NO: 96 - DNA, 318 bp, synthetic construct
  source 1..318: mol_type = other DNA
attgtgttgacacagtctccagccaccctgtctttgtctccaggggaaagagccaccctctcctgcagggccagtcagagtgttagcagctacttagcctggtatcaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctcccacttttggccaggggaccaagctggagatcaaa

SEQ ID NO: 97 - protein, 8 aa, synthetic construct
GFTFSNYA

SEQ ID NO: 98 - protein, 8 aa, synthetic construct
ISMSGGST

SEQ ID NO: 99 - protein, 10 aa, synthetic construct
AKDEYSSGWY

SEQ ID NO: 100 - protein, 121 aa, synthetic construct; identical to SEQ ID NO: 52

SEQ ID NO: 101 - DNA, 24 bp, synthetic construct
  source 1..24: mol_type = other DNA
ggattcacctttagcaactatgcc

SEQ ID NO: 102 - DNA, 24 bp, synthetic construct
  source 1..24: mol_type = other DNA
attagtatgagtggtggtagcaca

SEQ ID NO: 103 - DNA, 30 bp, synthetic construct
  source 1..30: mol_type = other DNA
gcgaaagacgaatatagcagtggctggtac

SEQ ID NO: 104 - DNA, 363 bp, synthetic construct; identical to SEQ ID NO: 56
  source 1..363: mol_type = other DNA

SEQ ID NO: 105 - protein, 6 aa, synthetic construct
QSVNSY

SEQ ID NO: 106 - skipped

SEQ ID NO: 107 - protein, 9 aa, synthetic construct
QQRSNWPPT

SEQ ID NO: 108 - protein, 107 aa, synthetic construct
EIVLTQSPATLSLSPGERATLSCRASQSVNSYLAWYQQKPGQAPRLLIYDASIRATGIPARFSGSGSGTDFTLTISSLEPEDFAFYYCQQRSNWPPTFGQGTKVEIK

SEQ ID NO: 109 - DNA, 18 bp, synthetic construct
  source 1..18: mol_type = other DNA
cagagtgttaacagctac

SEQ ID NO: 110 - skipped

SEQ ID NO: 111 - DNA, 27 bp, synthetic construct
  source 1..27: mol_type = other DNA
caacagcgtagtaactggcctccgacg

SEQ ID NO: 112 - DNA, 321 bp, synthetic construct
  source 1..321: mol_type = other DNA
gaaattgtgttgacacagtctccagccaccctgtctttgtctccaggggaaagagccaccctctcctgcagggccagtcagagtgttaacagctacttagcctggtaccaacagaaacctggccaggctcccaggctcctcatctatgatgcatccatcagggccactggcatcccagccaggttcagtggcagtgggtctgggacagacttcactctcaccatcagcagcctagagcctgaagattttgcattttattactgtcaacagcgtagtaactggcctccgacgttcggccaagggaccaaggtggaaatcaaa

SEQ ID NO: 113 - protein, 8 aa, synthetic construct
GYTFTSYW

SEQ ID NO: 114 - protein, 8 aa, synthetic construct
IDPNSGGT

SEQ ID NO: 115 - protein, 13 aa, synthetic construct
ARYDYYGSSYFDY

SEQ ID NO: 116 - protein, 120 aa, synthetic construct
QVQLQQPGAELVKPGASVKLSCKASGYTFTSYWMHWVKQRPGRGLEWIGRIDPNSGGTKYNEKFKSKATLTVDKPSSTAYMQLSSLTSEDSAVYYCARYDYYGSSYFDYWGQGTTVTVSS

SEQ ID NO: 117 - DNA, 24 bp, synthetic construct
  source 1..24: mol_type = other DNA
ggctacaccttcaccagctactgg

SEQ ID NO: 118 - DNA, 24 bp, synthetic construct
  source 1..24: mol_type = other DNA
atcgaccccaacagcggcggcaca

SEQ ID NO: 119 - DNA, 39 bp, synthetic construct
  source 1..39: mol_type = other DNA
gccagatacgactactacggcagctcttattttgattac

SEQ ID NO: 120 - DNA, 360 bp, synthetic construct
  source 1..360: mol_type = other DNA
caagtgcagctgcagcagcctggcgccgagctggtgaaacctggagcctccgtgaagctgagctgtaaggccagcggctacaccttcaccagctactggatgcactgggtcaagcagcggccaggaagaggcctggaatggatcggcagaatcgaccccaacagcggcggcacaaagtacaacgagaagttcaagagcaaggctacactgacagtggacaagcctagctccaccgcctacatgcagctgtctagcctgaccagcgaagatagcgccgtgtactactgcgccagatacgactactacggcagctcttattttgattactggggccagggcaccaccgtgaccgtgtccagc

SEQ ID NO: 121 - protein, 9 aa, synthetic construct
TGAVTTSNY

SEQ ID NO: 122 - skipped

SEQ ID NO: 123 - protein, 9 aa, synthetic construct
ALWYSNHWV

SEQ ID NO: 124 - protein, 109 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 125 - DNA, 27 bp, synthetic construct
  source 1..27: mol_type = other DNA
acaggcgctgtgaccacctctaactac

SEQ ID NO: 126 - skipped

SEQ ID NO: 127 - DNA, 27 bp, synthetic construct
  source 1..27: mol_type = other DNA
gccctgtggtacagcaatcactgggtg

SEQ ID NO: 128 - DNA, 327 bp, synthetic construct
  source 1..327: mol_type = other DNA
caagccgtggtgacacaggagagcgccctgaccacatcccccggcgagacagtgaccctgacctgtagaagcagcacaggcgctgtgaccacctctaactacgccaactgggtccaggaaaaacctgaccacctgtttaccggcctgatcggcggaaccaacaaccgggcccctggagtgccagccagattcagcggctctctgatcggcgacaaggccgctctcaccatcaccggcgcccagacagaagatgaggccatctacttctgcgccctgtggtacagcaatcactgggtgttcggcggaggcaccaagctgacagtgctg

SEQ ID NO: 129 - protein, 8 aa, synthetic construct
GYTFTDYY

SEQ ID NO: 130 - protein, 8 aa, synthetic construct
INPNNGGT

SEQ ID NO: 131 - protein, 13 aa, synthetic construct
ARNDGYRGYAMDY

SEQ ID NO: 132 - protein, 120 aa, synthetic construct
EVQLQQSGPELVKPGASVKISCKASGYTFTDYYMNWVKQSHGKSLEWIGGINPNNGGTSYNQKFKGKATLTVDKSSSTAYMELRSLTSEDSAVYYCARNDGYRGYAMDYWGQGTSVTVSS

SEQ ID NO: 133 - DNA, 24 bp, synthetic construct
  source 1..24: mol_type = other DNA
ggctacaccttcaccgattactac

SEQ ID NO: 134 - DNA, 24 bp, synthetic construct
  source 1..24: mol_type = other DNA
atcaaccccaacaacggcggaaca

SEQ ID NO: 135 - DNA, 39 bp, synthetic construct
  source 1..39: mol_type = other DNA
gccagaaatgatggctatagaggctacgccatggactac

SEQ ID NO: 136 - DNA, 360 bp, synthetic construct
  source 1..360: mol_type = other DNA
gaggtgcagctgcagcaatctggacctgagctggtgaagcctggcgcttctgtgaagatctcttgtaaagccagcggctacaccttcaccgattactacatgaactgggtcaagcagagccacggcaaaagcctggaatggatcggcggcatcaaccccaacaacggcggaacaagctacaaccagaagttcaagggcaaggctaccctgaccgtggacaagagcagctccaccgcctacatggaactgcggagcctgacaagcgaggacagcgccgtgtactactgcgccagaaatgatggctatagaggctacgccatggactactggggacagggcaccagcgtgacagtgtcctcc

SEQ ID NO: 137 - protein, 12 aa, synthetic construct
QSLLYSSNQKNY

SEQ ID NO: 138 - skipped

SEQ ID NO: 139 - protein, 10 aa, synthetic construct
QQYYSNPPLT

SEQ ID NO: 140 - protein, 114 aa, synthetic construct
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYCCQQYYSNPPLTFGAGTKLELK

SEQ ID NO: 141 - DNA, 36 bp, synthetic construct
  source 1..36: mol_type = other DNA
cagagcctgctgtacagcagcaaccagaaaaactac

SEQ ID NO: 142 - skipped

SEQ ID NO: 143 - DNA, 30 bp, synthetic construct
  source 1..30: mol_type = other DNA
cagcagtactacagcaatcctcctctgaca

SEQ ID NO: 144 - DNA, 342 bp, synthetic construct
  source 1..342: mol_type = other DNA
gacatcgtgatgagccagtctccatctagcctggccgtgtccgtgggcgagaaggtgaccatgtcttgtaaaagctcccagagcctgctgtacagcagcaaccagaaaaactacctggcctggtatcagcaaaagcccggccagagccccaagctcctgatctactgggcctccacacgggaaagcggagtgcctgatagattcaccggctctggcagcggaaccgactttaccctgaccatcagcagcgtcaaggccgaggacctggctgtgtactgctgccagcagtactacagcaatcctcctctgacattcggcgccggcacaaagctggaactgaag

SEQ ID NO: 145 - protein, 8 aa, synthetic construct
GFTFDDYA

SEQ ID NO: 146 - protein, 8 aa, synthetic construct
ITWNSGHI

SEQ ID NO: 147 - protein, 14 aa, synthetic construct
AKVSYLSTASSLDY

SEQ ID NO: 148 - protein, 121 aa, synthetic construct [sequence omitted]

SEQ ID NO: 149 - DNA, 24 bp, synthetic construct
  source 1..24: mol_type = other DNA
ggctttacattcgacgactacgcc

SEQ ID NO: 150 - DNA, 24 bp, synthetic construct
  source 1..24: mol_type = other DNA
atcacctggaatagcggccacatc

SEQ ID NO: 151 - DNA, 42 bp, synthetic construct
  source 1..42: mol_type = other DNA
gccaaagtgtcctacctgtctacagccagcagcctggactac

SEQ ID NO: 152 - DNA, 363 bp, synthetic construct
  source 1..363: mol_type = other DNA
gaggtgcagctggtggaaagcggaggaggcctggttcaacccggccggagcctgagactgagctgtgccgcttctggctttacattcgacgactacgccatgcactgggtcagacaggcccctggcaagggcctcgagtgggtgtccgccatcacctggaatagcggccacatcgactatgctgatagcgtggaaggcagattcaccatcagcagagataacgccaagaactccctgtacctgcagatgaacagcctgcgggccgaggacaccgccgtgtactactgcgccaaagtgtcctacctgtctacagccagcagcctggactactggggccagggcaccctggtgaccgtgtctagc

SEQ ID NO: 153 - protein, 6 aa, synthetic construct
QGISNY

SEQ ID NO: 154 - skipped

SEQ ID NO: 155 - protein, 9 aa, synthetic construct
HKYNSAPWT

SEQ ID NO: 156 - protein, 107 aa, synthetic construct
DIQMTQSPSSLSASVGDRVTITCRASQGISNYLAWYQQKPGQVPKLLIYAASALQSGVPSRFSGSGSGTDFTLTITSLQPEDVATYYCHKYNSAPWTFGQGTEVEIK

SEQ ID NO: 157 - DNA, 18 bp, synthetic construct
  source 1..18: mol_type = other DNA
cagggcatcagcaactac

SEQ ID NO: 158 - skipped

SEQ ID NO: 159 - DNA, 27 bp, synthetic construct
  source 1..27: mol_type = other DNA
cacaagtacaacagcgccccttggacc

SEQ ID NO: 160 - DNA, 321 bp, synthetic construct
  source 1..321: mol_type = other DNA
gacatccagatgacccagtcccctagctctctgagcgccagcgtgggcgaccgggtgaccatcacctgtagagcctctcagggcatcagcaactacctggcctggtatcagcagaaacccggacaggtgccaaagctgctgatctacgccgcttctgctctgcagagcggagtccccagcagattcagcggctccggcagcggcacagattttaccctgaccattacaagcctgcaacctgaggacgtggccacatactactgccacaagtacaacagcgccccttggaccttcggccagggcaccgaggtggaaatcaag

SEQ ID NO: 161 - protein, 8 aa, synthetic construct
GFTFTNYA

SEQ ID NO: 162 - protein, 8 aa, synthetic construct
ISGSGGRT

SEQ ID NO: 163 - protein, 16 aa, synthetic construct
AKDQTSGTTGYPYFAY

SEQ ID NO: 164 - protein, 123 aa, synthetic construct
ELQLLESGGGLVQPGGSLRLSCAASGFTFTNYAMNWVRQAPGKGLEWVSAISGSGGRTYYADSVKGRFTISRDNSRNTLFLQMNSLRPEDTAVYYCAKDQTSGTTGYPYFAYWGQGTLVTVSS

SEQ ID NO: 165 - DNA, 24 bp, synthetic construct
  source 1..24: mol_type = other DNA
ggctttacattcacaaattacgcc

SEQ ID NO: 166 - DNA, 24 bp, synthetic construct
  source 1..24: mol_type = other DNA
atctctggctccggcggcagaacc

SEQ ID NO: 167 - DNA, 48 bp, synthetic construct
  source 1..48: mol_type = other DNA
gccaaggatcagacatctggcacaacaggctacccttatttcgcctac

SEQ ID NO: 168 - DNA, 369 bp, synthetic construct
  source 1..369: mol_type = other DNA
gagctgcagctgctggagtctggcggcggcctggtgcagcccggcggctccctgaggctgagctgcgccgcctccggctttacattcacaaattacgccatgaattgggtgcggcaggccccaggcaagggcctggagtgggtgtccgccatctctggctccggcggcagaacctattacgccgattccgtgaagggcagattcaccatcagcagagacaactctcgcaataccctgtttctgcagatgaatagcctgaggcccgaggatacagccgtgtactactgcgccaaggatcagacatctggcacaacaggctacccttatttcgcctactggggccagggcaccctggtgaccgtgtcttcc

SEQ ID NO: 169 - protein, 6 aa, synthetic construct
QSVSSN

SEQ ID NO: 170 - skipped

SEQ ID NO: 171 - protein, 9 aa, synthetic construct
QQYNNWPRT

SEQ ID NO: 172 - protein, 107 aa, synthetic construct
ETVMTQSPATLSVSPGERAILSCRASQSVSSNLVWYQQKPGQAPRLFIYGASTRATGIPARFSGSGSGTEFTLTISSLQSEDFAVYYCQQYNNWPRTFGQGTKVEIK

SEQ ID NO: 173 - DNA, 18 bp, synthetic construct
  source 1..18: mol_type = other DNA
cagagcgtgtcttccaat

SEQ ID NO: 174 - skipped

SEQ ID NO: 175 - DNA, 27 bp, synthetic construct
  source 1..27: mol_type = other DNA
cagcagtataataactggcctaggacc

SEQ ID NO: 176 - DNA, 323 bp, synthetic construct
  source 1..323: mol_type = other DNA
gagacagtgatgacccagagcccagccaccctgtccgtgtctccaggcgagagggccatcctgtcctgtagggcctcccagagcgtgtcttccaatctggtgtggtaccagcagaagccaggccaggcccctaggctgtttatctacggcgcctccacaagggccaccggcatccctgccagattctctggctctggctccggcacagagttcaccctgacaatctcttccctgcagtctgaggactttgccgtgtactattgccagcagtataataactggcctaggacctttggccagggcacaaaggtggagatcaagca

SEQ ID NO: 177 - protein, 8 aa, synthetic construct
GFAFSSYG

SEQ ID NO: 178 - protein, 8 aa, synthetic construct
IWFDGTKK

SEQ ID NO: 179 - protein, 17 aa, synthetic construct
ARDRGIGARRGPYYMDV

SEQ ID NO: 180 - protein, 124 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 181 - DNA, 24 bp, synthetic construct
  source 1..24: mol_type = other DNA
ggatttgccttcagcagctacggc

SEQ ID NO: 182 - DNA, 24 bp, synthetic construct
  source 1..24: mol_type = other DNA
atctggttcgacggcacaaagaag

SEQ ID NO: 183 - DNA, 51 bp, synthetic construct
  source 1..51: mol_type = other DNA
gccagagatagaggcatcggcgccaggcggggaccttactacatggacgtg

SEQ ID NO: 184 - DNA, 372 bp, synthetic construct
  source 1..372: mol_type = other DNA
caagtgcagctggttgagagcggcggaggcgtggtgcagcccggcagaagcctgagactgtcttgtgctgcttctggatttgccttcagcagctacggcatgcactgggtgcggcaggcccctggcaagggcctggaatgggtcgccgtgatctggttcgacggcacaaagaagtactatacagacagcgtgaagggcagattcaccatcagcagagataatagcaagaacaccctgtacctgcagatgaacaccctgcgggccgaggacaccgccgtgtactactgcgccagagatagaggcatcggcgccaggcggggaccttactacatggacgtgtggggcaaaggcaccacagtgaccgtgtcctcc

SEQ ID NO: 185 - protein, 6 aa, synthetic construct
QSISSY

SEQ ID NO: 186 - skipped

SEQ ID NO: 187 - protein, 9 aa, synthetic construct
QQYYSTPLT

SEQ ID NO: 188 - protein, 107 aa, synthetic construct
DIQMTQSPSSLTASVGDRVTITCRASQSISSYVNWYQQKPGKAPKVLIFAASSLQSGVPSRFSGSGSGTDFTLTISSLQAEDVAVYYCQQYYSTPLTFGGGTKVEIK

SEQ ID NO: 189 - DNA, 18 bp, synthetic construct
  source 1..18: mol_type = other DNA
cagtccatcagctcctac

SEQ ID NO: 190 - skipped

SEQ ID NO: 191 - DNA, 27 bp, synthetic construct
  source 1..27: mol_type = other DNA
cagcaatactacagcacccctctgacc

SEQ ID NO: 192 - DNA, 321 bp, synthetic construct
  source 1..321: mol_type = other DNA
gatatccagatgacacagagccccagcagcctgaccgcctctgtgggcgaccgggtgaccatcacctgtagagccagccagtccatcagctcctacgtgaactggtatcagcagaaacctggcaaggcccctaaggtgctgatctttgctgccagcagcctgcagagcggagtcccatctagattcagcggctctggcagcggcaccgacttcaccctgacaattagctctctgcaggccgaggacgtggccgtgtactactgccagcaatactacagcacccctctgaccttcggcggcggaacaaaggtggaaatcaag

SEQ ID NO: 193 - protein, 8 aa, synthetic construct
GFLLISNG

SEQ ID NO: 194 - protein, 8 aa, synthetic construct
IWAGGNTN

SEQ ID NO: 195 - protein, 16 aa, synthetic construct
ARDFYDYDVFYYAMDY

SEQ ID NO: 196 - protein, 122 aa, synthetic construct
QVQLKESGPGLVAPSQSLSITCTVSGFLLISNGVHWVRQPPGKGLEWLGVIWAGGNTNYNSALMSRVSISKDNSKSQVFLKMKSLQTDDTAMYYCARDFYDYDVFYYAMDYWGQGTSVTVSS

SEQ ID NO: 197 - DNA, 24 bp, synthetic construct
  source 1..24: mol_type = other DNA
ggcttcctgctgatcagcaacggc

SEQ ID NO: 198 - DNA, 24 bp, synthetic construct
  source 1..24: mol_type = other DNA
atctgggctggcggaaataccaac

SEQ ID NO: 199 - DNA, 48 bp, synthetic construct
  source 1..48: mol_type = other DNA
gccagagatttctacgattatgacgttttttactacgccatggactac

SEQ ID NO: 200 - DNA, 423 bp, synthetic construct
  source 1..423: mol_type = other DNA
atgggctggtcctgtatcatcctgttcctggtcgccacagccaccggagtgcacagccaagtccagctgaaggaaagcggcccaggcctggtggcccctagccagagcctgagcatcacatgtaccgtgtccggcttcctgctgatcagcaacggcgtgcactgggtgcggcagcctcccggcaagggcctcgagtggctgggagtgatctgggctggcggaaataccaactacaacagcgccctgatgtctagagtgtctatctctaaggacaacagcaagtcccaggtgttcctgaagatgaaaagcctgcagaccgacgacaccgccatgtactactgcgccagagatttctacgattatgacgttttttactacgccatggactactggggccagggcaccagcgtgacagtgtccagc

SEQ ID NO: 201 - protein, 9 aa, synthetic construct
TGTVTTSNF

SEQ ID NO: 202 - skipped

SEQ ID NO: 203 - protein, 9 aa, synthetic construct
ALWYSNHFV

SEQ ID NO: 204 - protein, 109 aa, synthetic construct
QAVVTQESALTTSPGETVPLTCRSSTGTVTTSNFANWVQEKPDHLFTGLIGGTNNRAPGLPARFSGSLIGDKAALTITGAQTEDEAIFFCALWYSNHFVFGGGSQLTVL

SEQ ID NO: 205 - DNA, 27 bp, synthetic construct
  source 1..27: mol_type = other DNA
accggcaccgtgaccaccagcaacttc

SEQ ID NO: 206 - skipped

SEQ ID NO: 207 - DNA, 27 bp, synthetic construct
  source 1..27: mol_type = other DNA
gccctgtggtacagcaatcactttgtg

SEQ ID NO: 208 - DNA, 327 bp, synthetic construct
  source 1..327: mol_type = other DNA
caagccgtggtgacacaggaatccgccctcacaacctctccaggcgagacagtgcctctgacctgtagaagcagcaccggcaccgtgaccaccagcaacttcgccaactgggtccaggaaaagcccgaccacctgttcaccggcctgatcggcggaacaaacaaccgggcccctggcctgcctgctagattcagcggcagcctgatcggagataaggccgctctgacaatcaccggcgcccagaccgaggacgaggccatctttttctgcgccctgtggtacagcaatcactttgtgttcggcggcggatctcagctgaccgtgctg

(19) United States
(12) Patent Application Publication
(10) Pub. No.: US 2025/0215078 A1
(43) Pub. Date: Jul. 3, 2025

(54) Antibodies Against Human Siglec-9 and Use Thereof for Immunotherapy

(71) Applicant: The Wistar Institute of Anatomy and Biology, Philadelphia, PA (US)

(72) Inventors: David Weiner, Merion, PA (US); Devivasha Bordoloi, Philadelphia, PA (US); Kar Muthumani, Collegeville, PA (US); Mohamed Abdel-Mohsen

(21) Appl. No.: 18/852,652

(22) PCT Filed: Mar. 31, 2023
(86) PCT No.: PCT/US23/65215

§ 371 Date: Sep. 30, 2024

Related U.S. Application Data
(60) Provisional application No. 63/325,865, filed on Mar. 31, 2022.

(51) Int. Cl. C07K 16/28

(52) U.S. Cl. CPC C07K 16/2803; C07K 2317/24; C07K 2317/732

(57) ABSTRACT

The present invention provides compositions comprising anti-Siglec antibodies and nucleic acid molecules encoding the same, and methods for treating or preventing a disease or disorder using the same.

### CROSS REFERENCE TO RELATED APPLICATIONS

This application claims priority to U.S. Provisional Application No. 63/325,865, filed Mar. 31, 2022, which is hereby incorporated by reference herein in its entirety.

### BACKGROUND OF THE INVENTION

Cancers manipulate several immunological mechanisms to ensure a permissive local microenvironment that promotes tumor progression. Glycosylation is frequently cited as hallmark of cancer. Altered surface glycosylation of tumor/infected cells appears as a key feature of cancer and different infectious diseases. Sialylated glycans found on both glycoproteins and glycolipids are recognized by Siglecs, a family of lectins that are expressed on the surface of many immune cell subtypes. The interaction of cancer cells/infected cells bound sialic acid with Siglecs can thus modulate immune cell phenotype and allow them to escape the immune system.

Siglecs contain a Sia-binding N-terminal V-set domain followed by C2-set Ig-domains that are considered to act as spacers or regulators of oligomerisation. Majority of them function as transmembrane receptors in the immune system via recognition of Sia residues. Each type of Siglec prevalently recognizes a particular set of sialylated structures.

Siglec 9 is an important inhibitory sialic acid-binding immunoglobulin-like lectin (Siglec) receptors expressed by NK cells (CD16pos/CD56dim), B cells and monocytes. The interaction between Siglec 9 and Sia leads to the phosphorylation of immunoreceptor tyrosine-based inhibition motif (ITIM), which can suppress the cytotoxicity of NK cells.

Studies have shown that aberrantly glycosylated mucins including MUC 1 and MUC 16 on cancer cells bind to Siglec-9, on immune cells, to inhibit Natural killer (NK) and T cell anti-tumor function and modulate the immune functions of myeloid cells Siglec-9/sialic-acid binding may result in inhibitory signals that lead to compromised immune responses in the tumor microenvironment.

There remains a need in the art for novel cancer therapeutics that effectively treat cancer while minimizing negative effects. The present invention satisfies this unmet need.

### SUMMARY OF THE INVENTION

In one embodiment, the invention relates to an antibody or fragment thereof that specifically binds to a sialic acid-binding receptor. In one embodiment, the sialic acid-binding receptor is Siglec-9.

In one embodiment, the antibody comprises a variable heavy chain sequence comprising the CDR sequences of SEQ ID NO:1-3; SEQ ID NO:17-19; SEQ ID NO: 33-35; SEQ ID NO:49-51; SEQ ID NO:65-67; SEQ ID NO:81-83; SEQ ID NO: 97-99; SEQ ID NO:113-115; SEQ ID NO: 129-131; SEQ ID NO: 145-147; SEQ ID NO: 161-163; SEQ ID NO: 177-179; or SEQ ID NO: 193-195.

In one embodiment, the antibody comprises a variable heavy chain sequence comprising the CDR sequences of SEQ ID NO:9-11; SEQ ID NO:25-27; SEQ ID NO: 41-43; SEQ ID NO:57-59; SEQ ID NO: 73-75; SEQ ID NO: 89-91; SEQ ID NO: 105-107; SEQ ID NO: 121-123; SEQ ID NO: 137-139; SEQ ID NO: 153-155; SEQ ID NO: 169-171; SEQ ID NO:185-187; or SEQ ID NO:201-203.

In one embodiment, the antibody comprises a variable heavy chain sequence of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196.

In one embodiment, the antibody comprises a variable light chain sequence of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204.

In one embodiment, the antibody comprises a variable heavy chain sequence comprising the CDR sequences of SEQ ID NO:1-3 and a variable light chain sequence comprising the CDR sequences of SEQ ID NO:9-11. In one embodiment, the antibody comprises a variable heavy chain sequence comprising the CDR sequences of SEQ ID NO:17-19 and a variable light chain sequence comprising the CDR sequences of SEQ ID NO:25-27. In one embodiment, the antibody comprises a variable heavy chain sequence comprising the CDR sequences of SEQ ID NO:33-35 and a variable light chain sequence comprising the CDR sequences of SEQ ID NO:41-43. In one embodiment, the antibody comprises a variable heavy chain sequence comprising the CDR sequences of SEQ ID NO:49-51 and a variable light chain sequence comprising the CDR sequences of SEQ ID NO:57-59. In one embodiment, the antibody comprises a variable heavy chain sequence comprising the CDR sequences of SEQ ID NO:65-67 and a variable light chain sequence comprising the CDR sequences of SEQ ID NO:73-75. In one embodiment, the antibody comprises a variable heavy chain sequence comprising the CDR sequences of SEQ ID NO:81-83 and a variable light chain sequence comprising the CDR sequences of SEQ ID NO:89-91. In one embodiment, the antibody comprises a variable heavy chain sequence comprising the CDR sequences of SEQ ID NO:97-99 and a variable light chain sequence comprising the CDR sequences of SEQ ID NO:105-107. In one embodiment, the antibody comprises a variable heavy chain sequence comprising the CDR sequences of SEQ ID NO:113-115 and a variable light chain sequence comprising the CDR sequences of SEQ ID NO:121-123. In one embodiment, the antibody comprises a variable heavy chain sequence comprising the CDR sequences of SEQ ID NO: 129-131 and a variable light chain sequence comprising the CDR sequences of SEQ ID NO: 137-139. In one embodiment, the antibody comprises a variable heavy chain sequence comprising the CDR sequences of SEQ ID NO:145-147 and a variable light chain sequence comprising the CDR sequences of SEQ ID NO:153-155. In one embodiment, the antibody comprises a variable heavy chain sequence comprising the CDR sequences of SEQ ID NO: 161-163 and a variable light chain sequence comprising the CDR sequences of SEQ ID NO:169-171. In one embodiment, the antibody comprises a variable heavy chain sequence comprising the CDR sequences of SEQ ID NO:177-179 and a variable light chain sequence comprising the CDR sequences of SEQ ID NO:185-187. In one embodiment, the antibody comprises a variable heavy chain sequence comprising the CDR sequences of SEQ ID NO:193-195 and a variable light chain sequence comprising the CDR sequences of SEQ ID NO:201-203.

In one embodiment, the antibody comprises a variable heavy chain sequence of SEQ ID NO:4 and a variable light chain sequence of SEQ ID NO:12. In one embodiment, the antibody comprises a variable heavy chain sequence of SEQ ID NO:20 and a variable light chain sequence of SEQ ID NO:28. In one embodiment, the antibody comprises a variable heavy chain sequence of SEQ ID NO:36 and a variable light chain sequence of SEQ ID NO:44. In one embodiment, the antibody comprises a variable heavy chain sequence of SEQ ID NO:52 and a variable light chain sequence of SEQ ID NO: 60. In one embodiment, the antibody comprises a variable heavy chain sequence of SEQ ID NO:68 and a variable light chain sequence of SEQ ID NO:76. In one embodiment, the antibody comprises a variable heavy chain sequence of SEQ ID NO:84 and a variable light chain sequence of SEQ ID NO:92. In one embodiment, the antibody comprises a variable heavy chain sequence of SEQ ID NO:100 and a variable light chain sequence of SEQ ID NO:108. In one embodiment, the antibody comprises a variable heavy chain sequence of SEQ ID NO:116 and a variable light chain sequence of SEQ ID NO: 124. In one embodiment, the antibody comprises a variable heavy chain sequence of SEQ ID NO:132 and a variable light chain sequence of SEQ ID NO: 140. In one embodiment, the antibody comprises a variable heavy chain sequence of SEQ ID NO: 148 and a variable light chain sequence of SEQ ID NO:156. In one embodiment, the antibody comprises a variable heavy chain sequence of SEQ ID NO:164 and a variable light chain sequence of SEQ ID NO:172. In one embodiment, the antibody comprises a variable heavy chain sequence of SEQ ID NO: 180 and a variable light chain sequence of SEQ ID NO: 188. In one embodiment, the antibody comprises a variable heavy chain sequence of SEQ ID NO: 196 and a variable light chain sequence of SEQ ID NO:204.

In one embodiment, the antibody comprises a sequence having at least 95% identity to a variable heavy chain sequence of one or more of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196; a sequence having at least 95% identity to a variable light chain sequence of one or more of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204; a fragment comprising at least 80% of the full-length sequence of a variable heavy chain sequence of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196; or a fragment comprising at least 80% of the full-length of a variable light chain sequence of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204, or a combination thereof.

In one embodiment the invention relates to a nucleic acid molecule encoding an antibody or fragment thereof that specifically binds to a sialic acid-binding receptor. In one embodiment, the sialic acid-binding receptor is Siglec-9.

In one embodiment, the nucleic acid molecule encodes an antibody comprising a variable heavy chain sequence selected of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196; a variable light chain sequence of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204; a sequence having at least 95% identity to a variable heavy chain sequence of one or more of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196; a sequence having at least 95% identity to a variable light chain sequence of one or more of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204; a fragment comprising at least 80% of the full-length sequence of a variable heavy chain sequence of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196; or a fragment comprising at least 80% of the full-length of a variable light chain sequence selected from one or more of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204, or a combination thereof.

In some embodiments, the fragment of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196 comprises at least all three CDR regions of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196. In some embodiments, the variant of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196 comprises 100% identity to all three SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196.

In some embodiments, the fragment of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204 comprises at least all three CDR regions of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204. In some embodiments, the variant of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204 comprises 100% identity to all three CDR regions of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204.

In one embodiment, the nucleic acid molecule comprises nucleotide sequences encoding the variable heavy chain CDR sequences of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196. In one embodiment, the nucleic acid molecule comprises SEQ ID NO:5-7; SEQ ID NO:21-23; SEQ ID NO:37-39; SEQ ID NO:53-55; SEQ ID NO:69-71; SEQ ID NO:85-87; SEQ ID NO:101-103; SEQ ID NO:117-119; SEQ ID NO: 133-135; SEQ ID NO: 149-151; SEQ ID NO: 165-167; SEQ ID NO: 181-183; or SEQ ID NO:197-199. In one embodiment, the nucleic acid molecule comprises SEQ ID NO: 8; SEQ ID NO:24; SEQ ID NO:40; SEQ ID NO:56; SEQ ID NO:72; SEQ ID NO:88; SEQ ID NO:104; SEQ ID NO: 120; SEQ ID NO:136; SEQ ID NO:152; SEQ ID NO:168; SEQ ID NO:184; or SEQ ID NO:200. In one embodiment, the nucleic acid molecule comprises a nucleotide sequence having at least 95% identity to SEQ ID NO:8; SEQ ID NO: 24; SEQ ID NO:40; SEQ ID NO:56; SEQ ID NO:72; SEQ ID NO:88; SEQ ID NO: 104; SEQ ID NO: 120; SEQ ID NO: 136; SEQ ID NO: 152; SEQ ID NO: 168; SEQ ID NO: 184; or SEQ ID NO:200. In one embodiment, the nucleic acid molecule comprises a fragment comprising at least 80% of the full-length sequence of SEQ ID NO:8; SEQ ID NO: 24; SEQ ID NO:40; SEQ ID NO:56; SEQ ID NO:72; SEQ ID NO:88; SEQ ID NO: 104; SEQ ID NO: 120; SEQ ID NO: 136; SEQ ID NO: 152; SEQ ID NO:168; SEQ ID NO: 184; or SEQ ID NO:200.

In one embodiment, the nucleic acid molecule comprises nucleotide sequences encoding the variable light chain CDR sequences of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204. In one embodiment, the nucleic acid molecule comprises SEQ ID NO:13-15; SEQ ID NO:29-31; SEQ ID NO:45-47; SEQ ID NO:61-63; SEQ ID NO:77-79; SEQ ID NO:93-95; SEQ ID NO:109-111; SEQ ID NO:125-127; SEQ ID NO: 141-143; SEQ ID NO: 157-159; SEQ ID NO: 173-175; SEQ ID NO: 189-191; or SEQ ID NO:205-207. In one embodiment, the nucleic acid molecule comprises SEQ ID NO: 16; SEQ ID NO:32; SEQ ID NO:48; SEQ ID NO:64; SEQ ID NO:80; SEQ ID NO: 96; SEQ ID NO: 112; SEQ ID NO: 128; SEQ ID NO: 144; SEQ ID NO: 160; SEQ ID NO: 176; SEQ ID NO:192; or SEQ ID NO:208. In one embodiment, the nucleic acid molecule comprises a nucleotide sequence having at least 95% identity to SEQ ID NO: 16; SEQ ID NO:32; SEQ ID NO:48; SEQ ID NO:64; SEQ ID NO:80; SEQ ID NO: 96; SEQ ID NO:112; SEQ ID NO: 128; SEQ ID NO: 144; SEQ ID NO:160; SEQ ID NO: 176; SEQ ID NO:192; or SEQ ID NO:208. In one embodiment, the nucleic acid molecule comprises a fragment comprising at least 80% of the full-length sequence of SEQ ID NO:16; SEQ ID NO:32; SEQ ID NO:48; SEQ ID NO:64; SEQ ID NO:80; SEQ ID NO: 96; SEQ ID NO:112; SEQ ID NO: 128; SEQ ID NO: 144; SEQ ID NO: 160; SEQ ID NO: 176; SEQ ID NO:192; or SEQ ID NO:208.

In one embodiment, the invention provides a combination of nucleic acid molecules, wherein the first nucleic acid molecule comprises heavy chain CDR encoding sequences of SEQ ID NO:5-7; SEQ ID NO:21-23; SEQ ID NO:37-39; SEQ ID NO:53-55; SEQ ID NO:69-71; SEQ ID NO:85-87; SEQ ID NO:101-103; SEQ ID NO:117-119; SEQ ID NO: 133-135; SEQ ID NO: 149-151; SEQ ID NO: 165-167; SEQ ID NO:181-183; or SEQ ID NO:197-199 and the second nucleic acid molecule comprises light chain CDR encoding sequences of SEQ ID NO:13-15; SEQ ID NO:29-31; SEQ ID NO:45-47; SEQ ID NO: 61-63; SEQ ID NO: 77-79; SEQ ID NO:93-95; SEQ ID NO: 109-111; SEQ ID NO: 125-127; SEQ ID NO:141-143; SEQ ID NO: 157-159; SEQ ID NO: 173-175; SEQ ID NO: 189-191; or SEQ ID NO:205-207. In one embodiment, the combination of nucleic acid molecules comprises a first nucleic acid molecule comprising a heavy chain encoding sequence of SEQ ID NO:8; SEQ ID NO:24; SEQ ID NO:40; SEQ ID NO:56; SEQ ID NO:72; SEQ ID NO:88; SEQ ID NO: 104; SEQ ID NO:120; SEQ ID NO:136; SEQ ID NO:152; SEQ ID NO:168; SEQ ID NO:184; or SEQ ID NO:200 or a fragment or variant thereof, and a second nucleic acid molecule comprising a light chain encoding sequence of SEQ ID NO: 16; SEQ ID NO:32; SEQ ID NO:48; SEQ ID NO:64; SEQ ID NO: 80; SEQ ID NO:96; SEQ ID NO: 112; SEQ ID NO: 128; SEQ ID NO:144; SEQ ID NO: 160; SEQ ID NO: 176; SEQ ID NO: 192; or SEQ ID NO:208, or a fragment or variant thereof.

In one embodiment the invention relates to a composition comprising a sialic acid-binding receptor antibody. In one embodiment, the composition further comprises a pharmaceutically acceptable excipient. In one embodiment, the composition further comprises an adjuvant. In some embodiments, the composition further comprises a tumor antigen or a nucleotide sequence encoding a tumor antigen.

In one embodiment the invention relates to a composition comprising a nucleic acid molecule encoding a sialic acid-binding receptor antibody. In one embodiment, the nucleic acid molecule comprises an expression vector. In one embodiment, the nucleic acid molecule is incorporated into a viral particle. In one embodiment, the composition further comprises a pharmaceutically acceptable excipient. In one embodiment, the composition further comprises an adjuvant. In some embodiments, the composition further comprises a nucleotide sequence encoding a tumor antigen.

In one embodiment the invention relates to a method of treating or preventing a disease or disorder in a subject in need thereof, the method comprising administering a sialic acid-binding receptor antibody, a nucleic acid molecule encoding a sialic acid-binding receptor antibody, or a composition comprising a sialic acid-binding receptor antibody or a nucleic acid molecule encoding a sialic acid-binding receptor antibody. In one embodiment, the disease or disorder is a cancer, or a disease or disorder associated with cancer. In one embodiment, the disease or disorder is an infectious disease or disorder.

In one embodiment the invention relates to a method of increasing natural killer cell function in a subject in need thereof, the method comprising administering a sialic acid-binding receptor antibody, a nucleic acid molecule encoding a sialic acid-binding receptor antibody, or a composition comprising a sialic acid-binding receptor antibody or a nucleic acid molecule encoding a sialic acid-binding receptor antibody.

In one embodiment the invention relates to an immunogenic composition comprising an antibody or fragment thereof comprising that specifically binds to a sialic acid-binding receptor, and a nucleic acid molecule encoding a tumor antigen.

In one embodiment, the disease or disorder is a cancer, or a disease or disorder associated with cancer. In one embodiment, the cancer has increased levels of sialic acid. In one embodiment, the cancer is ovarian cancer, melanoma, renal cell carcinoma, prostate cancer, colon cancer, breast cancer, head and neck squamous cell carcinoma, or oral cancer.

In one embodiment the invention relates to a method of increasing natural killer cell function in a subject in need thereof, the method comprising administering an immunogenic composition comprising an antibody or fragment thereof that specifically binds to a sialic acid-binding receptor, and a nucleic acid molecule encoding a tumor antigen.

In one embodiment the invention relates to a method of increasing natural killer cell function in a subject in need thereof, the method comprising administering an immunogenic composition comprising a nucleic acid molecule encoding an antibody or fragment thereof that specifically binds to a sialic acid-binding receptor, and a nucleic acid molecule encoding a tumor antigen.

### BRIEF DESCRIPTION OF THE DRAWINGS

FIG. 1 shows binding of Anti-Siglec-9 clones to human Siglec 9. The specificity and binding activity of the generated hybridoma supernatants is shown.

FIG. 2 depicts exemplary experimental results demonstrating OVISE cell cytotoxicity by Siglec-9 antibody clones.

FIG. 3 depicts exemplary experimental results demonstrating OVCAR10 cell cytotoxicity by Siglec-9 antibody clones.

### DETAILED DESCRIPTION

The present invention relates to sialic acid receptor antibodies, fragments thereof, variants thereof, or a nucleic acid molecule encoding the same, and methods of use to increase the natural killer cell activity in a subject in need thereof.

In one aspect, the present invention relates to a composition that can be used to increase or enhance an immune response, i.e., create a more effective immune response, by administering sialic acid receptor antibodies, fragments thereof, variants thereof, or a nucleic acid molecule encoding the same. In one embodiment, the sialic acid receptor antibody is an antibody against Siglec-9.

In one aspect, the present invention relates to a combination of a sialic acid receptor antibody, fragment thereof, variant thereof, or a nucleic acid molecule encoding the same with a nucleic acid molecule encoding a tumor antigen.

In one aspect, the present invention relates to methods of treating a disease or disorder in a subject in need thereof, comprising administering to the subject a sialic acid receptor antibody, fragment thereof, variant thereof, or a nucleic acid molecule encoding the same. In one embodiment, the disease or disorder is cancer. In one embodiment, the disease or disorder is an infectious disease.

In one embodiment, the present invention relates to methods of treating cancer or a disease or disorder associated therewith in a subject in need thereof, comprising administering to the subject a sialic acid receptor antibody, fragment thereof, variant thereof, or a nucleic acid molecule encoding the same in combination with a nucleic acid molecule encoding a tumor antigen. In one embodiment, the cancer has increased levels of sialic acid.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope, or a sequence derived therefrom.

“Antigen” refers to proteins that have the ability to generate an immune response in a host. An antigen may be recognized and bound by an antibody. An antigen may originate from within the body or from the external environment.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Adjuvant” as used herein means any molecule added to the vaccine described herein to enhance the immunogenicity of the antigen.

“Coding sequence” or “encoding nucleic acid” as used herein may refer to the nucleic acid (RNA or DNA molecule) that comprise a nucleotide sequence which encodes an antibody as set forth herein. The coding sequence may also comprise a DNA sequence which encodes an RNA sequence. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may have Watson-Crick (e.g., A-T/U and C-G) or Hoogsteen base pairing between nucleotides or nucleotide analogs of nucleic acid molecules.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or CDNA.

An “effective amount” of a compound is that amount of compound which is sufficient to provide an effect to the subject or system to which the compound is administered.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The genetic construct may also refer to a DNA molecule which transcribes an RNA. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Identical” or “identity” as used herein in the context of two or more nucleic acids or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

The terms “patient,” “subject,” “individual,” and the like are used interchangeably herein, and refer to any animal, or cells thereof whether in vitro or in situ, amenable to the methods described herein. In some embodiments, the patient, subject or individual is a human.

“Parenteral” administration of a composition includes, e.g., subcutaneous (s.c.), intravenous (i.v.), intramuscular (i.m.), or intradermal injection, or infusion techniques.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV 40 late promoter and the CMV IE promoter.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein may facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering a vaccine of the present invention to a subject prior to onset of the disease. Suppressing the disease involves administering a vaccine of the present invention to a subject after induction of the disease but before its clinical appearance. Repressing the disease involves administering a vaccine of the present invention to a subject after clinical appearance of the disease.

A “therapeutic” treatment is a treatment administered to a subject who exhibits signs or symptoms of a disease or disorder, for the purpose of diminishing or eliminating the frequency or severity of those signs or symptoms.

As used herein, “treating a disease or disorder” means reducing the frequency or severity, or both, of at least one sign or symptom of the disease or disorder experienced by a patient.

The phrase “therapeutically effective amount,” as used herein, refers to an amount that is sufficient or effective to prevent or treat (delay or prevent the onset of, prevent the progression of, inhibit, decrease or reverse) a disease or disorder, including alleviating signs and/or symptoms of such diseases and disorders.

To “treat” a disease or disorder as the term is used herein, means to reduce the frequency or severity of at least one sign or symptom of a disease or disorder experienced by a subject.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of +2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Provided herein are antibodies or antibody-like molecules which specifically binds to Siglec-9. In one embodiment, the invention provides immunogenic compositions comprising the antibodies or antibody-like molecules which specifically binds to Siglec-9, a fragment thereof, a variant thereof, or a nucleic acid molecule encoding the same of the invention. The immunogenic compositions of the invention can be used to protect against diseases or disorders associated with an alteration in glycosylation, including, but not limited to, cancers and infectious disease.

Therefore, in some embodiments, the invention provides compositions comprising one or more antibody to Siglec-9, a fragment thereof, or a variant thereof, or a nucleic acid molecule encoding the same.

In some embodiments, the invention provides methods of treating or preventing a disease or disorder comprising administering to a subject an antibody or antibody-like molecule which specifically binds to Siglec-9, a fragment thereof, a variant thereof, or a nucleic acid molecule encoding the same of the invention.

In some embodiments, the invention provides methods of treating or preventing a cancer associated with increased Siglec-9 comprising administering to a subject an antibody or antibody-like molecule which specifically binds to Siglec-9, a fragment thereof, a variant thereof, or a nucleic acid molecule encoding the same of the invention.

### Antibody Compositions

In one embodiment, the invention relates to compositions comprising at least one Siglec-9 antibody, or a fragment or variant thereof.

In one embodiment, the anti-siglec-9 antibody, or fragment thereof comprises a heavy chain variable region having a sequence of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196, or a fragment or variant thereof. In one embodiment, the anti-siglec-9 antibody, or fragment thereof comprises a light chain variable region having a sequence of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204, or a fragment or variant thereof.

In one embodiment, the antibody comprises a variable heavy chain sequence comprising the CDR sequences of SEQ ID NO:1-3 and a variable light chain sequence comprising the CDR sequences of SEQ ID NO:9-11. In one embodiment, the antibody comprises a variable heavy chain sequence comprising the CDR sequences of SEQ ID NO:17-19 and a variable light chain sequence comprising the CDR sequences of SEQ ID NO:25-27. In one embodiment, the antibody comprises a variable heavy chain sequence comprising the CDR sequences of SEQ ID NO:33-35 and a variable light chain sequence comprising the CDR sequences of SEQ ID NO:41-43. In one embodiment, the antibody comprises a variable heavy chain sequence comprising the CDR sequences of SEQ ID NO:49-51 and a variable light chain sequence comprising the CDR sequences of SEQ ID NO:57-59. In one embodiment, the antibody comprises a variable heavy chain sequence comprising the CDR sequences of SEQ ID NO:65-67 and a variable light chain sequence comprising the CDR sequences of SEQ ID NO:73-75. In one embodiment, the antibody comprises a variable heavy chain sequence comprising the CDR sequences of SEQ ID NO:81-83 and a variable light chain sequence comprising the CDR sequences of SEQ ID NO:89-91. In one embodiment, the antibody comprises a variable heavy chain sequence comprising the CDR sequences of SEQ ID NO:97-99 and a variable light chain sequence comprising the CDR sequences of SEQ ID NO:105-107. In one embodiment, the antibody comprises a variable heavy chain sequence comprising the CDR sequences of SEQ ID NO:113-115 and a variable light chain sequence comprising the CDR sequences of SEQ ID NO:121-123. In one embodiment, the antibody comprises a variable heavy chain sequence comprising the CDR sequences of SEQ ID NO:129-131 and a variable light chain sequence comprising the CDR sequences of SEQ ID NO:137-139. In one embodiment, the antibody comprises a variable heavy chain sequence comprising the CDR sequences of SEQ ID NO:145-147 and a variable light chain sequence comprising the CDR sequences of SEQ ID NO:153-155. In one embodiment, the antibody comprises a variable heavy chain sequence comprising the CDR sequences of SEQ ID NO:161-163 and a variable light chain sequence comprising the CDR sequences of SEQ ID NO:169-171. In one embodiment, the antibody comprises a variable heavy chain sequence comprising the CDR sequences of SEQ ID NO: 177-179 and a variable light chain sequence comprising the CDR sequences of SEQ ID NO: 185-187. In one embodiment, the antibody comprises a variable heavy chain sequence comprising the CDR sequences of SEQ ID NO:193-195 and a variable light chain sequence comprising the CDR sequences of SEQ ID NO:201-203.

In one embodiment, the antibody comprises a variable heavy chain sequence of SEQ ID NO:4 and a variable light chain sequence of SEQ ID NO:12. In one embodiment, the antibody comprises a variable heavy chain sequence of SEQ ID NO:20 and a variable light chain sequence of SEQ ID NO:28. In one embodiment, the antibody comprises a variable heavy chain sequence of SEQ ID NO:36 and a variable light chain sequence of SEQ ID NO:44. In one embodiment, the antibody comprises a variable heavy chain sequence of SEQ ID NO:52 and a variable light chain sequence of SEQ ID NO: 60. In one embodiment, the antibody comprises a variable heavy chain sequence of SEQ ID NO:68 and a variable light chain sequence of SEQ ID NO:76. In one embodiment, the antibody comprises a variable heavy chain sequence of SEQ ID NO:84 and a variable light chain sequence of SEQ ID NO:92. In one embodiment, the antibody comprises a variable heavy chain sequence of SEQ ID NO:100 and a variable light chain sequence of SEQ ID NO:108. In one embodiment, the antibody comprises a variable heavy chain sequence of SEQ ID NO:116 and a variable light chain sequence of SEQ ID NO: 124. In one embodiment, the antibody comprises a variable heavy chain sequence of SEQ ID NO:132 and a variable light chain sequence of SEQ ID NO: 140. In one embodiment, the antibody comprises a variable heavy chain sequence of SEQ ID NO:148 and a variable light chain sequence of SEQ ID NO:156. In one embodiment, the antibody comprises a variable heavy chain sequence of SEQ ID NO:164 and a variable light chain sequence of SEQ ID NO:172. In one embodiment, the antibody comprises a variable heavy chain sequence of SEQ ID NO:180 and a variable light chain sequence of SEQ ID NO: 188. In one embodiment, the antibody comprises a variable heavy chain sequence of SEQ ID NO: 196 and a variable light chain sequence of SEQ ID NO:204.

In some embodiments, a variant of an amino acid sequence as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared to a defined amino acid sequence. In some embodiments, a variant of an amino acid sequence as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over the full length of variable heavy chain having an amino acid sequence of one or more of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196, or over the full length of variable light chain having an amino acid sequence of one or more of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204.

In some embodiments, the variant of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196 comprises 100% identity to all three CDR regions of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196. In some embodiments, the variant of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204 comprises 100% identity to all three CDR regions of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204.

As used herein, the term “antibody” or “immunoglobulin” refers to proteins (including glycoproteins) of the immunoglobulin (Ig) superfamily of proteins. An antibody or immunoglobulin (Ig) molecule may be tetrameric, comprising two identical light chain polypeptides and two identical heavy chain polypeptides. The two heavy chains are linked together by disulfide bonds, and each heavy chain is linked to a light chain by a disulfide bond. Each full-length Ig molecule contains at least two binding sites for a specific target or antigen.

A sialic acid-binding receptor antibody, or antigen-binding fragment thereof, includes, but is not limited to a polyclonal antibody, a monoclonal fusion proteins, antibodies or fragments thereof, chimerized or chimeric fusion proteins, antibodies or fragments thereof, humanized fusion proteins, antibodies or fragments thereof, deimmunized humfusion proteins, antibodies or fragments thereof, fully humfusion proteins, antibodies or fragments thereof, single chain antibody, single chain Fv fragment (scFv), Fv, Fd fragment, Fab fragment, Fab′ fragment, F (ab′) 2 fragment, diabody or antigen-binding fragment thereof, minibody or antigen-binding fragment thereof, triabody or antigen-binding fragment thereof, domain fusion proteins, antibodies or fragments thereof, camelid fusion proteins, antibodies or fragments thereof, dromedary fusion proteins, antibodies or fragments thereof, phage-displayed fusion proteins, antibodies or fragments thereof, or antibody, or antigen-binding fragment thereof, identified with a repetitive backbone array (e.g. repetitive antigen display).

The immune system produces several different classes of Ig molecules (isotypes), including IgA, IgD, IgE, IgG, and IgM, each distinguished by the particular class of heavy chain polypeptide present: alpha (a) found in IgA, delta (8) found in IgD, epsilon (¿) found in IgE, gamma (γ) found in IgG, and mu (u) found in IgM. There are at least five different γ heavy chain polypeptides (isotypes) found in IgG. In contrast, there are only two light chain polypeptide isotypes, referred to as kappa (κ) and lambda (2) chains. The distinctive characteristics of antibody isotypes are defined by sequences of the constant domains of the heavy chain.

An IgG molecule comprises two light chains (either κ or λ form) and two heavy chains (γ form) bound together by disulfide bonds. The κ and λ forms of IgG light chain each contain a domain of relatively variable amino acid sequences, called the variable region (variously referred to as a “V_{L}-,” “V_{κ}-,” or “V_{λ}-region”) and a domain of relatively conserved amino acid sequences, called the constant region (C_{L}-region). Similarly, each IgG heavy chain contains a variable region (V_{H}-region) and one or more conserved regions: a complete IgG heavy chain contains three constant domains (“C_{H}1-,” “C_{H}2-,” and “C_{H}3-regions”) and a hinge region. Within each V_{L}- or V_{H}-region, hypervariable regions, also known as complementarity-determining regions (“CDR”), are interspersed between relatively conserved framework regions (“FR”). Generally, the variable region of a light or heavy chain polypeptide contains four FRs and three CDRs arranged in the following order along the polypeptide: NH₂-FR1-CDR1-FR2-CDR2-FR3-CDR3-FR4-COOH. Together the CDRs and FRs determine the three-dimensional structure of the IgG binding site and thus, the specific target protein or antigen to which that IgG molecule binds. Each IgG molecule is dimeric, able to bind two antigen molecules. Cleavage of a dimeric IgG with the protease papain produces two identical antigen-binding fragments (“Fab”) and an “Fc” fragment or Fc domain, so named because it is readily crystallized.

As used throughout the present disclosure, the term “antibody” further refers to a whole or intact antibody (e.g., IgM, IgG, IgA, IgD, or IgE) molecule that is generated by any one of a variety of methods that are known in the art and described herein. The term “antibody” includes a polyclonal antibody, a monoclonal antibody, a chimerized or chimeric antibody, a humanized antibody, a deimmunized human antibody, and a fully human antibody. The antibody can be made in or derived from any of a variety of species, e.g., mammals such as humans, non-human primates (e.g., monkeys, baboons, or chimpanzees), horses, cattle, pigs, sheep, goats, dogs, cats, rabbits, guinea pigs, gerbils, hamsters, rats, and mice. The antibody can be a purified or a recombinant antibody.

As used herein, the term “epitope” refers to the site on a protein that is bound by an antibody. “Overlapping epitopes” include at least one (e.g., two, three, four, five, or six) common amino acid residue(s).

In one embodiment, the antibody of the invention specifically binds to a Siglec polypeptide. As used herein, the terms “specific binding” or “specifically binds” refer to two molecules forming a complex that is relatively stable under physiologic conditions. Typically, binding is considered specific when the association constant (Kₐ) is higher than 10⁶ M-1. Thus, an antibody can specifically bind to a target with a Kₐ of at least (or greater than) 10⁶ (e.g., at least or greater than 10⁷, 10⁸, 10⁹, 10¹⁰, 10¹¹, 10¹², 10¹³, 10¹⁴, or 10¹⁵ or higher) M⁻¹.

In one embodiment, the antibody of the invention specifically binds to Siglec-9.

Methods for determining whether an antibody binds to a protein antigen and/or the affinity for an antibody to a protein antigen are known in the art. For example, the binding of an antibody to a protein antigen can be detected and/or quantified using a variety of techniques such as, but not limited to, Western blot, dot blot, surface plasmon resonance method (e.g., BIAcore system; Pharmacia Biosensor AB, Uppsala, Sweden and Piscataway, N.J.), or enzyme-linked immunosorbent assays (ELISA). See, e.g., Harlow and Lane (1988) “Antibodies: A Laboratory Manual” Cold Spring Harbor Laboratory Press, Cold Spring Harbor, N.Y.; Benny K. C. Lo (2004) “Antibody Engineering: Methods and Protocols,” Humana Press (ISBN: 1588290921); Borrebaek (1992) “Antibody Engineering, A Practical Guide,” W.H. Freeman and Co., NY; Borrebaek (1995) “Antibody Engineering,” 2nd Edition, Oxford University Press, NY, Oxford; Johne et al. (1993) J. Immunol. Meth. 160:191-198; Jonsson et al. (1993) Ann. Biol. Clin. 51:19-26; and Jonsson et al. (1991) Biotechniques 11:620-627. See also, U.S. Pat. No. 6,355,245.

Immunoassays which can be used to analyze immunospecific binding and cross-reactivity of the antibodies include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, fluorescent immunoassays, and protein A immunoassays. Such assays are routine and well known in the art.

Antibodies can also be assayed using any surface plasmon resonance (SPR)-based assays known in the art for characterizing the kinetic parameters of the interaction of the antibody with its target or epitope. Any SPR instrument commercially available including, but not limited to, BIAcore Instruments (Biacore AB; Uppsala, Sweden); 1Asys instruments (Affinity Sensors; Franklin, Massachusetts); IBIS system (Windsor Scientific Limited; Berks, UK), SPR-CELLIA systems (Nippon Laser and Electronics Lab; Hokkaido, Japan), and SPR Detector Spreeta (Texas Instruments; Dallas, Texas) can be used in the methods described herein. See, e.g., Mullett et al. (2000) Methods 22:77-91; Dong et al. (2002) Reviews in Mol Biotech 82:303-323; Fivash et al. (1998) Curr Opin Biotechnol 9:97-101; and Rich et al. (2000) Curr Opin Biotechnol 11:54-61.

The antibodies and fragments thereof can be, in some embodiments, “chimeric.” Chimeric antibodies and antigen-binding fragments thereof comprise portions from two or more different species (e.g., mouse and human). Chimeric antibodies can be produced with mouse variable regions of desired specificity spliced onto human constant domain gene segments (see, for example, U.S. Pat. No. 4,816,567). In this manner, non-human antibodies can be modified to make them more suitable for human clinical application (e.g., methods for treating or preventing a complement associated disorder in a human subject).

The monoclonal antibodies of the present disclosure include “humanized” forms of the non-human (e.g., mouse) antibodies. Humanized or CDR-grafted mAbs are particularly useful as therapeutic agents for humans because they are not cleared from the circulation as rapidly as mouse antibodies and do not typically provoke an adverse immune reaction. Methods of preparing humanized antibodies are generally well known in the art. For example, humanization can be essentially performed following the method of Winter and co-workers (see, e.g., Jones et al. (1986) Nature 321:522-525; Riechmann et al. (1988) Nature 332:323-327; and Verhoeyen et al. (1988) Science 239:1534-1536), by substituting rodent CDRs or CDR sequences for the corresponding sequences of a human antibody. Also see, e.g., Staelens et al. (2006) Mol Immunol 43:1243-1257. In some embodiments, humanized forms of non-human (e.g., mouse) antibodies are human antibodies (recipient antibody) in which hypervariable (CDR) region residues of the recipient antibody are replaced by hypervariable region residues from a non-human species (donor antibody) such as a mouse, rat, rabbit, or non-human primate having the desired specificity, affinity, and binding capacity. In some instances, framework region residues of the human immunoglobulin are also replaced by corresponding non-human residues (so called “back mutations”). In addition, phage display libraries can be used to vary amino acids at chosen positions within the antibody sequence. The properties of a humanized antibody are also affected by the choice of the human framework. Furthermore, humanized and chimerized antibodies can be modified to comprise residues that are not found in the recipient antibody or in the donor antibody in order to further improve antibody properties, such as, for example, affinity or effector function.

Fully human antibodies are also provided in the disclosure. The term “human antibody” includes antibodies having variable and constant regions (if present) derived from human germline immunoglobulin sequences. Human antibodies can include amino acid residues not encoded by human germline immunoglobulin sequences (e.g., mutations introduced by random or site-specific mutagenesis in vitro or by somatic mutation in vivo). However, the term “human antibody” does not include antibodies in which CDR sequences derived from the germline of another mammalian species, such as a mouse, have been grafted onto human framework sequences (i.e., humanized antibodies). Fully human or human antibodies may be derived from transgenic mice carrying human antibody genes (carrying the variable (V), diversity (D), joining (J), and constant (C) exons) or from human cells. For example, it is now possible to produce transgenic animals (e.g., mice) that are capable, upon immunization, of producing a full repertoire of human antibodies in the absence of endogenous immunoglobulin production. (See, e.g., Jakobovits et al. (1993) Proc. Natl. Acad. Sci. USA 90:2551; Jakobovits et al. (1993) Nature 362:255-258; Bruggemann et al. (1993) Year in Immunol. 7:33; and Duchosal et al. (1992) Nature 355:258.) Transgenic mice strains can be engineered to contain gene sequences from unrearranged human immunoglobulin genes. The human sequences may code for both the heavy and light chains of human antibodies and would function correctly in the mice, undergoing rearrangement to provide a wide antibody repertoire similar to that in humans. The transgenic mice can be immunized with the target protein (to create a diverse array of specific antibodies and their encoding RNA. Nucleic acids encoding the antibody chain components of such antibodies may then be cloned from the animal into a display vector. Typically, separate populations of nucleic acids encoding heavy and light chain sequences are cloned, and the separate populations then recombined on insertion into the vector, such that any given copy of the vector receives a random combination of a heavy and a light chain. The vector is designed to express antibody chains so that they can be assembled and displayed on the outer surface of a display package containing the vector. For example, antibody chains can be expressed as fusion proteins with a phage coat protein from the outer surface of the phage. Thereafter, display packages can be screened for display of antibodies binding to a target.

Thus, in some embodiments, the disclosure provides, e.g., humanized, deimmunized or primatized antibodies comprising one or more of the complementarity determining regions (CDRs) of the mouse monoclonal antibodies described herein, which retain the ability (e.g., at least 50, 60, 70, 80, 90, or 100%, or even greater than 100%) of the mouse monoclonal antibody counterpart to bind to its antigen.

In addition, human antibodies can be derived from phage-display libraries (Hoogenboom et al. (1991) J. Mol. Biol. 227:381; Marks et al. (1991) J. Mol. Biol, 222:581-597; and Vaughan et al. (1996) Nature Biotech 14:309 (1996)). Synthetic phage libraries can be created which use randomized combinations of synthetic human antibody V-regions. By selection on antigen fully human antibodies can be made in which the V-regions are very human-like in nature. See, e.g., U.S. Pat. Nos. 6,794,132, 6,680,209, 4,634,666, and Ostberg et al. (1983), Hybridoma 2:361-367, the contents of each of which are incorporated herein by reference in their entirety.

For the generation of human antibodies, also see Mendez et al. (1998) Nature Genetics 15:146-156 and Green and Jakobovits (1998) J. Exp. Med. 188:483-495, the disclosures of which are hereby incorporated by reference in their entirety. Human antibodies are further discussed and delineated in U.S. Pat. Nos. 5,939,598; 6,673,986; 6,114,598; 6,075,181; 6,162,963; 6,150,584; 6,713,610; and 6,657,103 as well as U.S. Patent Application Publication Nos. 2003-0229905 Al, 2004-0010810 Al, US 2004-0093622 Al, 2006-0040363 Al, 2005-0054055 Al, 2005-0076395 Al, and 2005-0287630 Al. See also International Publication Nos. WO 94/02602, WO 96/34096, and WO 98/24893, and European Patent No. EP 0 463 151 B1. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in, e.g., U.S. Pat. Nos. 5,545,807; 5,545,806; 5,625,825; 5,625,126; 5,633,425; 5,661,016; 5,770,429; 5,789,650; and U.S. Pat. Nos. 5,814,318; 5,591,669; 5,612,205; 5,721,367; 5,789,215; 5,643,763; 5,569,825; 5,877,397; 6,300,129; 5,874,299; 6,255,458; and 7,041,871, the disclosures of which are hereby incorporated by reference. See also European Patent No. 0 546 073 B1, International Patent Publication Nos. WO 92/03918, WO 92/22645, WO 92/22647, WO 92/22670, WO 93/12227, WO 94/00569, WO 94/25585, WO 96/14436, WO 97/13852, and WO 98/24884, the disclosures of each of which are hereby incorporated by reference in their entirety. See further Taylor et al. (1992) Nucleic Acids Res. 20:6287; Chen et al. (1993) Int. Immunol. 5:647; Tuaillon et al. (1993) Proc. Natl. Acad. Sci. USA 90:3720-4; Choi et al. (1993) Nature Genetics 4:1 17; Lonberg et al. (1994) Nature 368:856-859; Taylor et al. (1994) International Immunology 6:579-591; Tuaillon et al. (1995) J. Immunol. 154:6453-65; Fishwild et al. (1996) Nature Biotechnology 14:845; and Tuaillon et al. (2000) Eur. J. Immunol. 10:2998-3005, the disclosures of each of which are hereby incorporated by reference in their entirety.

In some embodiments, de-immunized antibodies or antigen-binding fragments thereof are provided. De-immunized antibodies or antigen-binding fragments thereof are antibodies that have been modified so as to render the antibody or antigen-binding fragment thereof non-immunogenic, or less immunogenic, to a given species (e.g., to a human). De-immunization can be achieved by modifying the fusion proteins, antibodies or fragments thereof utilizing any of a variety of techniques known to those skilled in the art (see, e.g., PCT Publication Nos. WO 04/108158 and WO 00/34317). For example, fusion proteins, antibodies or fragments thereof may be de-immunized by identifying potential T cell epitopes and/or B cell epitopes within the amino acid sequence of the fusion proteins, antibodies or fragments thereof and removing one or more of the potential T cell epitopes and/or B cell epitopes from the fusion proteins, antibodies or fragments thereof, for example, using recombinant techniques. The modified antibody or antigen-binding fragment thereof may then optionally be produced and tested to identify antibodies or antigen-binding fragments thereof that have retained one or more desired biological activities, such as, for example, binding affinity, but have reduced immunogenicity. Methods for identifying potential T cell epitopes and/or B cell epitopes may be carried out using techniques known in the art, such as, for example, computational methods (see e.g., PCT Publication No. WO 02/069232), in vitro or in silico techniques, and biological assays or physical methods (such as, for example, determination of the binding of peptides to MHC molecules, determination of the binding of peptide: MHC complexes to the T cell receptors from the species to receive the fusion proteins, antibodies or fragments thereof, testing of the protein or peptide parts thereof using transgenic animals with the MHC molecules of the species to receive the antibody or antigen-binding fragment thereof, or testing with transgenic animals reconstituted with immune system cells from the species to receive the fusion proteins, antibodies or fragments thereof, etc.). In various embodiments, the de-immunized antibodies described herein include de-immunized antigen-binding fragments, Fab, Fv, scFv, Fab′ and F (ab′) 2, monoclonal antibodies, murine antibodies, engineered antibodies (such as, for example, chimeric, single chain, CDR-grafted, humanized, fully human antibodies, and artificially selected antibodies), synthetic antibodies and semi-synthetic antibodies.

In some embodiments, the present disclosure also provides bispecific antibodies. Bispecific antibodies are monoclonal, preferably human or humanized, antibodies that have binding specificities for at least two different antigens. For example, in one embodiment, a bispecific antibody of the invention comprises one domain with a binding specificity for a Siglec protein or polypeptide, and one domain with a binding specificity for an alternative protein or polypeptide. In one embodiment, a bispecific antibody of the invention comprises one domain with a binding specificity for a Siglec protein or polypeptide, and one domain with a binding specificity for an alternative Siglec protein or polypeptide.

Methods for making bispecific antibodies are within the purview of those skilled in the art. Traditionally, the recombinant production of bispecific antibodies is based on the co-expression of two immunoglobulin heavy-chain/light-chain pairs, where the two heavy chain/light-chain pairs have different specificities (Milstein and Cuello (1983) Nature 305:537-539). Antibody variable domains with the desired binding specificities (antibody-antigen combining sites) can be fused to immunoglobulin constant domain sequences. The fusion of the heavy chain variable region is preferably with an immunoglobulin heavy-chain constant domain, including at least part of the hinge, CH2, and CH3 regions. DNAs encoding the immunoglobulin heavy-chain fusions and, if desired, the immunoglobulin light chain, are inserted into separate expression vectors, and are co-transfected into a suitable host organism. For further details of illustrative currently known methods for generating bispecific antibodies see, e.g., Suresh et al. (1986) Methods in Enzymology 121:210; PCT Publication No. WO 96/27011; Brennan et al. (1985) Science 229:81; Shalaby et al, J Exp Med (1992) 175:217-225; Kostelny et al. (1992) J Immunol 148 (5): 1547-1553; Hollinger et al. (1993) Proc Natl Acad Sci USA 90:6444-6448; Gruber et al. (1994) J Immunol 152:5368; and Tutt et al. (1991) J Immunol 147:60. Bispecific antibodies also include cross-linked or hetero-conjugate antibodies. Hetero-conjugate antibodies may be made using any convenient cross-linking methods. Suitable cross-linking agents are well known in the art, and are disclosed in U.S. Pat. No. 4,676,980, along with a number of cross-linking techniques.

Various techniques for making and isolating bispecific antibody fragments directly from recombinant cell culture have also been described. For example, bispecific antibodies have been produced using leucine zippers. See, e.g., Kostelny et al. (1992) J Immunol 148 (5): 1547-1553. The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers. This method can also be utilized for the production of antibody homodimers. The “diabody” technology described by Hollinger et al. (1993) Proc Natl Acad Sci USA 90:6444-6448 has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (scFv) dimers has also been reported. See, e.g., Gruber et al. (1994) J Immunol 152:5368. Alternatively, the antibodies can be “linear antibodies” as described in, e.g., Zapata et al. (1995) Protein Eng. 8 (10): 1057-1062. Briefly, these antibodies comprise a pair of tandem Fd segments (VH-CH1-VH-CH1) which form a pair of antigen binding regions. Linear antibodies can be bispecific or monospecific.

Antibodies with more than two valencies (e.g., trispecific antibodies) are also contemplated and described in, e.g., Tutt et al. (1991) J Immunol 147:60.

The disclosure also embraces variant forms of multi-specific antibodies such as the dual variable domain immunoglobulin (DVD-lg) molecules described in Wu et al. (2007) Nat Biotechnol 25 (11): 1290-1297. The DVD-lg molecules are designed such that two different light chain variable domains (VL) from two different parent antibodies are linked in tandem directly or via a short linker by recombinant DNA techniques, followed by the light chain constant domain. Similarly, the heavy chain comprises two different heavy chain variable domains (VH) linked in tandem, followed by the constant domain CH1 and Fc region. Methods for making DVD-Ig molecules from two parent antibodies are further described in, e.g., PCT Publication Nos. WO 08/024188 and WO 07/024715.

The disclosure also provides camelid or dromedary antibodies (e.g., antibodies derived from Camelus bactrianus, Calelus dromaderius, or Lama paccos). Such antibodies, unlike the typical two-chain (fragment) or four-chain (whole antibody) antibodies from most mammals, generally lack light chains. See U.S. Pat. No. 5,759,808; Stijlemans et al. (2004) J Biol Chem 279:1256-1261; Dumoulin et al. (2003) Nature 424:783-788; and Pleschberger et al. (2003) Bioconjugate Chem 14:440-448.

Engineered libraries of camelid antibodies and antibody fragments are commercially available, for example, from Ablynx (Ghent, Belgium). As with other antibodies of non-human origin, an amino acid sequence of a camelid antibody can be altered recombinantly to obtain a sequence that more closely resembles a human sequence, i.e., the nanobody can be “humanized” to thereby further reduce the potential immunogenicity of the antibody.

In some embodiments, the present disclosure also provides antibodies, or antigen-binding fragments thereof, which are variants of a peptide, protein or antibody described herein. In some embodiments, such a variant peptide, protein or antibody maintains the binding or inhibitory ability of the parent peptide, protein or antibody. Methods to prepare variants of known proteins, peptides or antibodies are known in the art. In some embodiments, such a variant comprises at least a single amino acid substitution, deletion, insertion, or other modification. In some embodiments, fusion proteins, antibodies or fragments thereof described herein comprises two or more (e.g. 2, 3, 4, 5, 6, 7, 8, 9, 10, 11, 12, 13, 14, 15, 16, 17, 18, 19, 20 or more) amino acid modifications (e.g., amino acid substitutions, deletions, or additions). In some embodiments, fusion proteins, antibodies or fragments thereof described herein does not contain an amino acid modification in a CDR. In some embodiments, fusion proteins, antibodies or fragments thereof described herein does contain one or more (e.g. 1, 2, 3, 4, 5, 6, 7, 8, 9, 10, 11, 12, 13, 14, 15, 16, 17, 18, 19, or 20) amino acid modifications in a CDR.

As used herein, the term “antibody fragment”, “antigen-binding fragment”, “antigen binding fragment”, or similar terms refer to fragment of an antibody that retains the ability to bind to an antigen wherein the antigen binding fragment may optionally include additional compositions not part of the original antibody (e.g. different framework regions or mutations) as well as the fragment(s) from the original antibody. Examples include, but are not limited to, a single chain antibody, a single chain Fv fragment (scFv), an Fd fragment, an Fab fragment, an Fab′ fragment, or an F (ab′) 2 fragment. An scFv fragment is a single polypeptide chain that includes both the heavy and light chain variable regions of the antibody from which the scFv is derived. In addition, diabodies (Poljak (1994) Structure 2 (12): 1121-1123; Hudson et al. (1999) J. Immunol. Methods 23 (1-2): 177-189, the disclosures of each of which are incorporated herein by reference in their entirety), minibodies, triabodies (Schoonooghe et al. (2009) BMC Biotechnol 9:70), and domain antibodies (also known as “heavy chain immunoglobulins” or camelids; Holt et al. (2003) Trends Biotechnol 21 (1 1): 484-490), (the disclosures of each of which are incorporated herein by reference in their entirety) that bind to a complement component protein can be incorporated into the compositions, and used in the methods, described herein. In some embodiments, any of the antigen binding fragments described herein may be included under “antigen binding fragment thereof or equivalent terms, when referring to fragments related to an antibody, whether such fragments were actually derived from the antibody or are antigen binding fragments that bind the same epitope or an overlapping epitope or an epitope contained in the antibody's epitope. An antigen binding fragment thereof may include antigen-binding fragments that bind the same, or overlapping, antigen as the original antibody and wherein the antigen binding fragment includes a portion (e.g. one or more CDRs, one or more variable regions, etc.) that is a fragment of the original antibody.

In some embodiments, the fragment of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196 comprises at least all three CDR regions of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196. In some embodiments, the fragment of SEQ ID NO: 12, SEQ ID NO: 28, SEQ ID NO: 44, SEQ ID NO: 60, SEQ ID NO: 76, SEQ ID NO: 92, SEQ ID NO: 10⁸, SEQ ID NO: 124, SEQ ID NO: 140, SEQ ID NO: 156, SEQ ID NO: 172, SEQ ID NO: 188, or SEQ ID NO: 204 comprises at least all three CDR regions of SEQ ID NO: 12, SEQ ID NO: 28, SEQ ID NO: 44, SEQ ID NO: 60, SEQ ID NO: 76, SEQ ID NO: 92, SEQ ID NO: 10⁸, SEQ ID NO: 124, SEQ ID NO: 140, SEQ ID NO: 156, SEQ ID NO: 172, SEQ ID NO: 188, or SEQ ID NO: 204. In some embodiments, the fragment of the sialic acid-binding receptor comprises an scFv antibody fragment that comprises a heavy chain of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196, or all three CDR regions of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196 and a light chain of SEQ ID NO: 12, SEQ ID NO: 28, SEQ ID NO: 44, SEQ ID NO: 60, SEQ ID NO: 76, SEQ ID NO: 92, SEQ ID NO: 10⁸, SEQ ID NO: 124, SEQ ID NO: 140, SEQ ID NO: 156, SEQ ID NO: 172, SEQ ID NO: 188, or SEQ ID NO: 204 or all three CDR regions of SEQ ID NO: 12, SEQ ID NO: 28, SEQ ID NO: 44, SEQ ID NO: 60, SEQ ID NO: 76, SEQ ID NO: 92, SEQ ID NO: 10⁸, SEQ ID NO: 124, SEQ ID NO: 140, SEQ ID NO: 156, SEQ ID NO: 172, SEQ ID NO: 188, or SEQ ID NO: 204.

In some embodiments, the antibodies described herein comprise an altered or mutated sequence that leads to altered stability or half-life compared to parent antibodies. This includes, for example, an increased stability or half-life for higher affinity or longer clearance time in vitro or in vivo, or a decreased stability or half-life for lower affinity or quicker removal. Additionally, the antibodies described herein may contain one or more (e.g. 1, 2, 3, 4, 5, 6, 7, 8, 9, 10, 11, 12, 13, 14, 15, 16, 17, 18, 19, or 20) amino acid substitutions, deletions, or insertions that result in altered post-translational modifications, including, for example, an altered glycosylation pattern (e.g., the addition of one or more sugar components, the loss of one or more sugar components, or a change in composition of one or more sugar components.

In some embodiments, the antibodies described herein comprise reduced (e.g. or no) effector function. Altered effector functions include, for example, a modulation in one or more of the following activities: antibody-dependent cellular cytotoxicity (ADCC), complement-dependent cytotoxicity (CDC), apoptosis, binding to one or more Fc-receptors, and pro-inflammatory responses. Modulation refers to an increase, decrease, or elimination of an effector function activity exhibited by a subject antibody containing an altered constant region as compared to the activity of the unaltered form of the constant region. In particular embodiments, modulation includes situations in which an activity is abolished or completely absent.

Antibodies with altered or no effector functions may be generated by engineering or producing antibodies with variant constant, Fc, or heavy chain regions; recombinant DNA technology and/or cell culture and expression conditions may be used to produce antibodies with altered function and/or activity. For example, recombinant DNA technology may be used to engineer one or more amino acid substitutions, deletions, or insertions in regions (such as, for example, Fc or constant regions) that affect antibody function including effector functions. Alternatively, changes in post-translational modifications, such as, e.g., glycosylation patterns, may be achieved by manipulating the cell culture and expression conditions by which the antibody is produced. Suitable methods for introducing one or more substitutions, additions, or deletions into an Fc region of an antibody are well known in the art and include, e.g., standard DNA mutagenesis techniques as described in, e.g., Sambrook et al. (1989) “Molecular Cloning: A Laboratory Manual, 2nd Edition,” Cold Spring Harbor Laboratory Press, Cold Spring Harbor, N.Y.; Harlow and Lane (1988), supra; Borrebaek (1992), supra; Johne et al. (1993), supra; PCT publication no. WO 06/53301; and U.S. Pat. No. 7,704,497.

### Nucleic Acid Molecules

Provided herein are polynucleotides that encode the Siglec-9 antibodies, or fragments thereof, of the invention. In some embodiments, the polynucleotide also comprises a sequence encoding a signal peptide operably linked at the 5′ end of the encoding sequence. In some embodiments, the polynucleotide also comprises a sequence encoding a linker sequence.

In one embodiment, the nucleic acid molecule encodes an antibody comprising a variable heavy chain sequence selected of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196; a variable light chain sequence of SEQ ID NO: 12, SEQ ID NO: 28, SEQ ID NO: 44, SEQ ID NO: 60, SEQ ID NO: 76, SEQ ID NO: 92, SEQ ID NO: 10⁸, SEQ ID NO: 124, SEQ ID NO: 140, SEQ ID NO: 156, SEQ ID NO: 172, SEQ ID NO: 188, or SEQ ID NO: 204; a sequence having at least 95% identity to a variable heavy chain sequence of one or more of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196; a sequence having at least 95% identity to a variable light chain sequence of one or more of SEQ ID NO: 12, SEQ ID NO: 28, SEQ ID NO: 44, SEQ ID NO: 60, SEQ ID NO: 76, SEQ ID NO: 92, SEQ ID NO: 10⁸, SEQ ID NO: 124, SEQ ID NO: 140, SEQ ID NO: 156, SEQ ID NO: 172, SEQ ID NO: 188, or SEQ ID NO: 204; a fragment comprising at least 80% of the full-length sequence of a variable heavy chain sequence of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196; or a fragment comprising at least 80% of the full-length of a variable light chain sequence selected from one or more of SEQ ID NO: 12, SEQ ID NO: 28, SEQ ID NO: 44, SEQ ID NO: 60, SEQ ID NO: 76, SEQ ID NO: 92, SEQ ID NO: 10⁸, SEQ ID NO: 124, SEQ ID NO: 140, SEQ ID NO: 156, SEQ ID NO: 172, SEQ ID NO: 188, or SEQ ID NO: 204, or a combination thereof.

In one embodiment, the nucleic acid molecule comprises nucleotide sequences encoding the variable heavy chain CDR sequences of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196. In one embodiment, the nucleic acid molecule comprises SEQ ID NO:5-7; SEQ ID NO:21-23; SEQ ID NO:37-39; SEQ ID NO:53-55; SEQ ID NO:69-71; SEQ ID NO:85-87; SEQ ID NO: 101-103; SEQ ID NO:117-119; SEQ ID NO: 133-135; SEQ ID NO: 149-151; SEQ ID NO: 165-167; SEQ ID NO: 181-183; or SEQ ID NO: 197-199. In one embodiment, the nucleic acid molecule comprises SEQ ID NO: 8; SEQ ID NO:24; SEQ ID NO:40; SEQ ID NO:56; SEQ ID NO:72; SEQ ID NO:88; SEQ ID NO: 104; SEQ ID NO: 120; SEQ ID NO:136; SEQ ID NO:152; SEQ ID NO:168; SEQ ID NO:184; or SEQ ID NO:200.

In one embodiment, the nucleic acid molecule comprises nucleotide sequences encoding the variable light chain CDR sequences of SEQ ID NO: 12, SEQ ID NO: 28, SEQ ID NO: 44, SEQ ID NO: 60, SEQ ID NO: 76, SEQ ID NO: 92, SEQ ID NO: 10⁸, SEQ ID NO: 124, SEQ ID NO: 140, SEQ ID NO: 156, SEQ ID NO: 172, SEQ ID NO: 188, or SEQ ID NO: 204. In one embodiment, the nucleic acid molecule comprises SEQ ID NO: 13-15; SEQ ID NO:29-31; SEQ ID NO:45-47; SEQ ID NO:61-63; SEQ ID NO:77-79; SEQ ID NO:93-95; SEQ ID NO:10⁹-111; SEQ ID NO:125-127; SEQ ID NO: 141-143; SEQ ID NO:157-159; SEQ ID NO: 173-175; SEQ ID NO:189-191; or SEQ ID NO:205-207. In one embodiment, the nucleic acid molecule comprises SEQ ID NO: 16; SEQ ID NO:32; SEQ ID NO:48; SEQ ID NO:64; SEQ ID NO:80; SEQ ID NO: 96; SEQ ID NO: 112; SEQ ID NO: 128; SEQ ID NO: 144; SEQ ID NO: 160; SEQ ID NO: 176; SEQ ID NO: 192; or SEQ ID NO:208.

In some embodiments, a variant of a nucleotide sequence as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared to a defined nucleotide sequence. In some embodiments, a variant of a nucleotide sequence as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over the full length of a nucleotide sequence. In some embodiments, the variant of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196 comprises 100% identity to all three SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196. In some embodiments, the variant of SEQ ID NO: 12, SEQ ID NO: 28, SEQ ID NO: 44, SEQ ID NO: 60, SEQ ID NO: 76, SEQ ID NO: 92, SEQ ID NO: 10⁸, SEQ ID NO: 124, SEQ ID NO: 140, SEQ ID NO: 156, SEQ ID NO: 172, SEQ ID NO: 188, or SEQ ID NO: 204 comprises 100% identity to all three CDR regions of SEQ ID NO: 12, SEQ ID NO: 28, SEQ ID NO: 44, SEQ ID NO: 60, SEQ ID NO: 76, SEQ ID NO: 92, SEQ ID NO: 10⁸, SEQ ID NO: 124, SEQ ID NO: 140, SEQ ID NO: 156, SEQ ID NO: 172, SEQ ID NO: 188, or SEQ ID NO: 204. Therefore, in some embodiments, the variability is in a region outside of a CDR-encoding region.

In one embodiment, the nucleic acid molecule comprises a nucleotide sequence having at least 95% identity to SEQ ID NO:8; SEQ ID NO:24; SEQ ID NO:40; SEQ ID NO:56; SEQ ID NO:72; SEQ ID NO:88; SEQ ID NO:104; SEQ ID NO:120; SEQ ID NO: 136; SEQ ID NO:152; SEQ ID NO:168; SEQ ID NO:184; or SEQ ID NO: 200. In one embodiment, the nucleic acid molecule comprises a nucleotide sequence having at least 95% identity to SEQ ID NO: 16; SEQ ID NO:32; SEQ ID NO:48; SEQ ID NO: 64; SEQ ID NO:80; SEQ ID NO:96; SEQ ID NO: 112; SEQ ID NO: 128; SEQ ID NO: 144; SEQ ID NO:160; SEQ ID NO:176; SEQ ID NO: 192; or SEQ ID NO:208.

In some embodiments, a fragment of a nucleotide sequence as described herein comprises at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full-length sequence of a defined nucleotide sequence. In some embodiments, a fragment of a nucleotide sequence as described herein comprises at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full length nucleotide sequence. In some embodiments, the fragment of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196 comprises at least all three CDR regions of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196. In some embodiments, the fragment of SEQ ID NO: 12, SEQ ID NO: 28, SEQ ID NO: 44, SEQ ID NO: 60, SEQ ID NO: 76, SEQ ID NO: 92, SEQ ID NO: 10⁸, SEQ ID NO: 124, SEQ ID NO: 140, SEQ ID NO: 156, SEQ ID NO: 172, SEQ ID NO: 188, or SEQ ID NO: 204 comprises at least all three CDR regions of SEQ ID NO: 12, SEQ ID NO: 28, SEQ ID NO: 44, SEQ ID NO: 60, SEQ ID NO: 76, SEQ ID NO: 92, SEQ ID NO: 10⁸, SEQ ID NO: 124, SEQ ID NO: 140, SEQ ID NO: 156, SEQ ID NO: 172, SEQ ID NO: 188, or SEQ ID NO: 204.

In one embodiment, the nucleic acid molecule comprises a fragment comprising at least 80% of the full-length sequence of SEQ ID NO:8; SEQ ID NO:24; SEQ ID NO:40; SEQ ID NO:56; SEQ ID NO:72; SEQ ID NO:88; SEQ ID NO:104; SEQ ID NO: 120; SEQ ID NO:136; SEQ ID NO:152; SEQ ID NO:168; SEQ ID NO:184; or SEQ ID NO:200. In one embodiment, the nucleic acid molecule comprises a fragment comprising at least 80% of the full-length sequence of SEQ ID NO:16; SEQ ID NO:32; SEQ ID NO:48; SEQ ID NO:64; SEQ ID NO:80; SEQ ID NO:96; SEQ ID NO:112; SEQ ID NO: 128; SEQ ID NO:144; SEQ ID NO:160; SEQ ID NO:176; SEQ ID NO:192; or SEQ ID NO:208.

In one embodiment, the invention provides a combination of nucleic acid molecules, wherein the first nucleic acid molecule comprises heavy chain CDR encoding sequences of SEQ ID NO:5-7; SEQ ID NO:21-23; SEQ ID NO:37-39; SEQ ID NO:53-55; SEQ ID NO:69-71; SEQ ID NO:85-87; SEQ ID NO:101-103; SEQ ID NO:117-119; SEQ ID NO: 133-135; SEQ ID NO:149-151; SEQ ID NO: 165-167; SEQ ID NO: 181-183; or SEQ ID NO:197-199 and the second nucleic acid molecule comprises light chain CDR encoding sequences of SEQ ID NO: 13-15; SEQ ID NO:29-31; SEQ ID NO:45-47; SEQ ID NO: 61-63; SEQ ID NO:77-79; SEQ ID NO:93-95; SEQ ID NO: 10⁹-111; SEQ ID NO: 125-127; SEQ ID NO: 141-143; SEQ ID NO: 157-159; SEQ ID NO: 173-175; SEQ ID NO: 189-191; or SEQ ID NO:205-207. In one embodiment, the combination of nucleic acid molecules comprises a first nucleic acid molecule comprising a heavy chain encoding sequence of SEQ ID NO:8; SEQ ID NO:24; SEQ ID NO:40; SEQ ID NO:56; SEQ ID NO:72; SEQ ID NO:88; SEQ ID NO:104; SEQ ID NO:120; SEQ ID NO:136; SEQ ID NO:152; SEQ ID NO:168; SEQ ID NO:184; or SEQ ID NO:200 or a fragment or variant thereof, and a second nucleic acid molecule comprising a light chain encoding sequence of SEQ ID NO: 16; SEQ ID NO:32; SEQ ID NO:48; SEQ ID NO:64; SEQ ID NO: 80; SEQ ID NO:96; SEQ ID NO:112; SEQ ID NO: 128; SEQ ID NO: 144; SEQ ID NO: 160; SEQ ID NO:176; SEQ ID NO:192; or SEQ ID NO:208, or a fragment or variant thereof.

The isolated nucleic acid may comprise any type of nucleic acid, including, but not limited to DNA, cDNA, and RNA. For example, in one embodiment, the composition comprises an isolated DNA molecule, including for example, an isolated cDNA molecule, encoding a protein inhibitor or functional fragment thereof. In one embodiment, the composition comprises an isolated RNA molecule encoding a protein inhibitor or a functional fragment thereof.

In some embodiments, the invention provides a combination of nucleic acid molecules comprising a first nucleic acid molecule comprising a nucleotide sequence encoding an antibody heavy chain and a second nucleic acid molecule encoding an antibody light chain. Therefore, in some embodiments, the invention provides a combination of a first nucleic acid molecule comprising a nucleotide sequence encoding a variable heavy chain of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, or SEQ ID NO: 196 and a second nucleic acid molecule encoding a variable light chain of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, or SEQ ID NO: 204.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides the phosphoester group connecting to adjacent ribonucleotides is replaced by a modified group, e.g., of phosphothioate group.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino) propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. The above modifications may be combined.

In some instances, the nucleic acid molecule comprises at least one of the following chemical modifications: 2′-H, 2′-O-methyl, or 2′-OH modification of one or more nucleotides. In some embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target. In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, for example as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, for example different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone. Modifications of the nucleic acid of the invention may be present at one or more of, a phosphate group, a sugar group, backbone, N-terminus, C-terminus, or nucleobase.

The present invention also includes a vector in which the isolated nucleic acid of the present invention is inserted. The art is replete with suitable vectors that are useful in the present invention.

Therefore, in another aspect, the invention relates to a vector, comprising the nucleotide sequence of the invention or the construct of the invention. The choice of the vector will depend on the host cell in which it is to be subsequently introduced. In some embodiments, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

In some embodiments, the expression of synthetic nucleic acids encoding a protein is typically achieved by operably linking a nucleic acid encoding the protein or portions thereof to a promoter and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and an IgE signal peptide.

The vectors of the present invention may also be used for nucleic acid immunization, using standard gene delivery protocols. Methods for gene delivery are known in the art. See, e.g., U.S. Pat. Nos. 5,399,346, 5,580,859, 5,589,466, incorporated by reference herein in their entireties.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In one embodiment, the recombinant expression vectors may also contain nucleic acid molecules, which encode a peptide or protein of invention, described elsewhere herein.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

In some embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of transformed or transfected host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin, such as IgG. The selectable markers may be introduced on a separate vector from the nucleic acid of interest.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In order to assess the expression of a protein inhibitor, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a peptide or protein into a host cell include calcium phosphate precipitation, lipofection, particle bombardment, microinjection, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York).

Biological methods for introducing a peptide or protein of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a peptide or protein into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### ScFv Antibody

In one embodiment, the antibody fragment comprises an scFv fragment. In one embodiment, the ScFv antibody fragment relates to a Fab fragment without the CH1 and CL regions. Thus, in one embodiment, the scFv antibody fragment relates to a Fab fragment comprising the VH and VL. In one embodiment, the scFv antibody fragment comprises a linker between VH and VL. In one embodiment, the scFv antibody fragment comprises the VH, VL and the CH2 and CH3 regions. In one embodiment, the scFv antibody fragment of the invention has modified expression, stability, half-life, antigen binding, heavy chain-light chain pairing, tissue penetration or a combination thereof as compared to a parental MAb.

In one embodiment, the scFv antibody fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher expression than the parental MAb.

In one embodiment, the scFv antibody fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher antigen binding than the parental MAb.

In one embodiment, the scFv antibody fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold longer half-life than the parental MAb.

In one embodiment, the scFv antibody fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher stability than the parental MAb.

In one embodiment, the scFv antibody fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater tissue penetration than the parental MAb.

In one embodiment, the scFv antibody fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater heavy chain-light chain pairing than the parental MAb.

### Delivery Vehicles

In one embodiment, the present invention provides a composition comprising a delivery vehicle comprising a sialic acid-binding receptor antibody, fragment thereof, or nucleic acid molecule encoding the same, as described herein.

Exemplary delivery vehicles include, but are not limited to, microspheres, microparticles, nanoparticles, polymerosomes, liposomes, and micelles. For example, in some embodiments, the delivery vehicle is loaded with an anti-Siglec-9 polypeptide, fragment thereof, or nucleic acid molecule encoding the same. In some embodiments, the delivery vehicle provides for controlled release, delayed release, or continual release of its loaded cargo. In some embodiments, the delivery vehicle comprises a targeting moiety that targets the delivery vehicle to a treatment site.

### Pharmaceutical Compositions

The present invention also provides pharmaceutical compositions comprising one or more of the compositions described herein. Formulations may be employed in admixtures with conventional excipients, i.e., pharmaceutically acceptable organic or inorganic carrier substances suitable for administration to a treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

Administration of the compositions of this invention may be carried out, for example, by parenteral, by intravenous, subcutaneous, intramuscular, or intraperitoneal injection, or by infusion or by any other acceptable systemic method.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group: benzyl alcohol, sorbic acid, parabens, imidurea and combinations thereof.

In one embodiment, the composition includes an anti-oxidant and a chelating agent that inhibits the degradation of one or more components of the composition. Exemplary antioxidants for some compounds are BHT, BHA, alpha-tocopherol and ascorbic acid. Exemplary chelating agents include edetate salts (e.g. disodium edetate) and citric acid. The chelating agent is useful for chelating metal ions in the composition that may be detrimental to the shelf life of the formulation. While BHT and disodium edetate may be the antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the compounds or other compositions of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para hydroxybenzoates, ascorbic acid, and sorbic acid.

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, chewing gum, varnishes, sealants, oral and teeth “dissolving strips”, or an emulsion. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compositions of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY—P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Excipients and Other Components of the Composition

The composition may further comprise a pharmaceutically acceptable excipient. The pharmaceutically acceptable excipient can be functional molecules such as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include transfection facilitating agents such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant in addition to the checkpoint inhibitor antibodies of the invention. The additional adjuvant can be other genes that are expressed in an alternative plasmid or are delivered as proteins in combination with the plasmid above in the composition. The adjuvant may be selected from the group consisting of: α-interferon (IFN-α), β-interferon (IFN-β), y-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, PD-1, IL-10, IL-12, IL-18, or a combination thereof.

Other genes that can be useful as adjuvants in addition to the antibodies of the invention include those encoding: MCP-1, MIP-la, MIP-1p, IL-8, RANTES, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The composition may further comprise a genetic facilitator agent as described in U.S. Ser. No. 021,579 filed Apr. 1, 1994, which is fully incorporated by reference.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligrams. In some preferred embodiments, composition according to the present invention comprises about 5 nanogram to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### Methods of Administration

The present invention provides a method for increasing a function or activity of natural killer (NK) cells. This can be measured for example in a standard NK- or T-cell based cytotoxicity assay, in which the capacity of a therapeutic compound to stimulate killing of sialic-acid ligand positive cells by Siglec positive lymphocytes is measured. In one embodiment, an antibody preparation causes at least a 10% augmentation in the cytotoxicity of a Siglec-restricted lymphocyte, optionally at least a 40% or 50% augmentation in lymphocyte cytotoxicity, or optionally at least a 70% augmentation in NK cytotoxicity, and referring to the cytotoxicity assays described. In one embodiment, an antibody preparation causes at least a 10% augmentation in cytokine release by a Siglec-restricted lymphocyte, optionally at least a 40% or 50% augmentation in cytokine release, or optionally at least a 70% augmentation in cytokine release, and referring to the cytotoxicity assays described. In one embodiment, an antibody preparation causes at least a 10% augmentation in cell surface expression of a marker of cytotoxicity (e.g. CD107 and/or CD137) by a Siglec-restricted lymphocyte, optionally at least a 40% or 50% augmentation, or optionally at least a 70% augmentation in cell surface expression of a marker of cytotoxicity (e.g. CD107 and/or CD137).

The present invention is also directed to a method of increasing an immune response in a subject. Increasing the immune response can be used to treat and/or prevent disease in the subject. The method can include administering the herein disclosed vaccine to the subject. The subject administered the vaccine can have an increased or boosted immune response as compared to a subject administered the antigen alone. In some embodiments, the immune response can be increased by about 0.5-fold to about 15-fold, about 0.5-fold to about 10-fold, or about 0.5-fold to about 8-fold. Alternatively, the immune response in the subject administered the vaccine can be increased by at least about 0.5-fold, at least about 1.0-fold, at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, or at least about 15.0-fold.

In still other alternative embodiments, the immune response in the subject administered the vaccine can be increased about 50% to about 1500%, about 50% to about 1000%, or about 50% to about 800%. In other embodiments, the immune response in the subject administered the vaccine can be increased by at least about 50%, at least about 100%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1050%, at least about 1100%, at least about 1150%, at least about 1200%, at least about 1250%, at least about 1300%, at least about 1350%, at least about 1450%, or at least about 1500%.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

### Combination Vaccine

In one embodiment, the invention relates to the administration of a Siglec-9 antibody of the invention, or nucleic acid molecule encoding the same, in combination with one or more additional therapeutic agent. In some embodiments, one or more additional therapeutic agent may be used to induce or increase an immune response. In some embodiments therefore, the invention relates to immunogenic compositions, such as vaccines, comprising a Siglec-9 antibody of the invention, or nucleic acid molecule encoding the same, in combination with an antigenic protein, a fragment thereof, or a variant thereof, a nucleic acid molecule encoding the same, or a combination thereof. In some embodiments therefore, the invention relates to immunogenic compositions, such as vaccines, comprising a Siglec-9 antibody of the invention, or nucleic acid molecule encoding the same, in combination with a PD-(L)1 axis inhibitor, a fragment thereof, or a variant thereof, a nucleic acid molecule encoding the same, or a combination thereof.

The combination vaccine can significantly induce an immune response of a subject administered the vaccine, thereby protecting against or treating a disease or disorder.

The immunogenic composition can be a DNA vaccine, a peptide vaccine, or a combination DNA and peptide vaccine. The DNA vaccine can include a nucleic acid sequence encoding an antigen. The nucleic acid sequence can be DNA, RNA, cDNA, a variant thereof, a fragment thereof, or a combination thereof. The nucleic acid sequence can also include additional sequences that encode linker, leader, or tag sequences that are linked to the antigen by a peptide bond. The peptide vaccine can include an antigenic peptide, an antigenic protein, a variant thereof, a fragment thereof, or a combination thereof. The combination DNA and peptide vaccine can include the above described nucleic acid sequence encoding the antigen and the antigenic peptide or protein.

The vaccine can induce a humoral immune response in the subject administered the vaccine. The induced humoral immune response can be specific for the antigen. The induced humoral immune response can be reactive with the antigen. The humoral immune response can be induced in the subject administered the vaccine by about 1.5-fold to about 16-fold, about 2-fold to about 12-fold, or about 3-fold to about 10-fold. The humoral immune response can be induced in the subject administered the vaccine by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold.

The humoral immune response induced by the vaccine can include an increased level of neutralizing antibodies associated with the subject administered the vaccine as compared to a subject not administered the vaccine. The neutralizing antibodies can be specific for the antigen. The neutralizing antibodies can be reactive with an antigen. The neutralizing antibodies can provide protection against and/or treatment of a disease or disorder in the subject administered the vaccine.

The humoral immune response induced by the vaccine can include an increased level of IgG antibodies associated with the subject administered the vaccine as compared to a subject not administered the vaccine. These IgG antibodies can be specific for the tumor antigen. These IgG antibodies can be reactive with the antigen. The level of IgG antibody associated with the subject administered the vaccine can be increased by about 1.5-fold to about 16-fold, about 2-fold to about 12-fold, or about 3-fold to about 10-fold as compared to the subject not administered the vaccine. The level of IgG antibody associated with the subject administered the vaccine can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to the subject not administered the vaccine.

The vaccine can induce a cellular immune response in the subject administered the vaccine. The induced cellular immune response can be specific for the antigen. The induced cellular immune response can be reactive to the antigen. The induced cellular immune response can include eliciting a CD8⁺ T cell response. The elicited CD8⁺ T cell response can be reactive with the antigen. The elicited CD8⁺ T cell response can be polyfunctional. The induced cellular immune response can include eliciting a CD8⁺ T cell response, in which the CD8⁺ T cells produce interferon-gamma (IFN-γ), tumor necrosis factor alpha (TNF-α), interleukin-2 (IL-2), or a combination of IFN-γ and TNF-α.

The induced cellular immune response can include an increased CD8⁺ T cell response associated with the subject administered the vaccine as compared to the subject not administered the vaccine. The CD8⁺ T cell response associated with the subject administered the vaccine can be increased by about 2-fold to about 30-fold, about 3-fold to about 25-fold, or about 4-fold to about 20-fold as compared to the subject not administered the vaccine. The CD8⁺ T cell response associated with the subject administered the vaccine can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to the subject not administered the vaccine.

The induced cellular immune response can include an increased frequency of CD3⁺CD8⁺ T cells that produce IFN-γ. The frequency of CD3⁺CD8⁺IFN-γ⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the vaccine.

The induced cellular immune response can include an increased frequency of CD3⁺CD8⁺ T cells that produce TNF-α. The frequency of CD3⁺CD8⁺ TNF-α⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, or 14-fold as compared to the subject not administered the vaccine.

The induced cellular immune response can include an increased frequency of CD3⁺CD8⁺ T cells that produce IL-2. The frequency of CD3⁺CD8⁺IL-2⁺ T cells associated with the subject administered the vaccine can be increased by at least about 0.5-fold, 1.0-fold, 1.5-fold, 2.0-fold, 2.5-fold, 3.0-fold, 3.5-fold, 4.0-fold, 4.5-fold, or 5.0-fold as compared to the subject not administered the vaccine.

The induced cellular immune response can include an increased frequency of CD3⁺CD8⁺ T cells that produce both IFN-γ and TNF-α. The frequency of CD3⁺CD8⁺IFN-γTNF-α⁺ T cells associated with the subject administered the vaccine can be increased by at least about 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold, 150-fold, 160-fold, 170-fold, or 180-fold as compared to the subject not administered the vaccine.

The cellular immune response induced by the vaccine can include eliciting a CD4⁺ T cell response. The elicited CD4⁺ T cell response can be reactive with the antigen. The elicited CD4⁺ T cell response can be polyfunctional. The induced cellular immune response can include eliciting a CD4⁺ T cell response, in which the CD4⁺ T cells produce IFN-γ, TNF-α, IL-2, or a combination of IFN-γ and TNF-α.

The induced cellular immune response can include an increased frequency of CD3⁺CD4⁺ T cells that produce IFN-γ. The frequency of CD3⁺CD4⁺IFN-γ⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the vaccine.

The induced cellular immune response can include an increased frequency of CD3⁺CD4⁺ T cells that produce TNF-α. The frequency of CD3⁺CD4⁺ TNF-α⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, or 22-fold as compared to the subject not administered the vaccine.

The induced cellular immune response can include an increased frequency of CD3⁺CD4⁺ T cells that produce IL-2. The frequency of CD3⁺CD4⁺IL-2⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 45-fold, 50-fold, 55-fold, or 60-fold as compared to the subject not administered the vaccine.

The induced cellular immune response can include an increased frequency of CD3⁺CD4⁺ T cells that produce both IFN-γ and TNF-α. The frequency of CD3⁺CD4⁺IFN-γ⁺TNF-α⁺ associated with the subject administered the vaccine can be increased by at least about 2-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 22-fold, 23-fold 24-fold, 25-fold, 26-fold, 27-fold, 28-fold, 29-fold, 30-fold, 31-fold, 32-fold, 33-fold, 34-fold, or 35-fold as compared to the subject not administered the vaccine.

The vaccine of the present invention can have features required of effective vaccines such as being safe so the vaccine itself does not cause illness or death; is protective against illness resulting from exposure to live pathogens such as viruses or bacteria; induces neutralizing antibody to prevent invention of cells; induces protective T cells against intracellular pathogens; and provides ease of administration, few side effects, biological stability, and low cost per dose.

The vaccine can further induce an immune response when administered to different tissues such as the muscle or skin.

### Tumor Antigen

As described above, in some embodiments, the combination vaccine of the invention comprises an anti-Siglec-9 antibody or nucleic acid molecule encoding the same in combination with a tumor antigen, a fragment thereof, a variant thereof, a nucleic acid molecule encoding the same, or a combination thereof.

Therefore, in some embodiments, the present invention includes compositions for enhancing an immune response against an antigen in a subject in need thereof, comprising a sialic acid-binding receptor antibody of the invention in combination with a synthetic antigen capable of generating an immune response in the subject, or a biologically functional fragment or variant thereof, or a nucleic acid molecule encoding the same.

In one embodiment, the antigen is a tumor associated surface antigen. Illustrative examples of a tumor associated surface antigen are CD10, CD19, CD20, CD22, CD33, Fms-like tyrosine kinase 3 (FLT-3, CD135), chondroitin sulfate proteoglycan 4 (CSPG4, melanoma-associated chondroitin sulfate proteoglycan), Epidermal growth factor receptor (EGFR), Her2neu, Her3, IGFR, CD133, IL3R, fibroblast activating protein (FAP), CDCP1, Derlin1, Tenascin, frizzled 1-10, the vascular antigens VEGFR2 (KDR/FLK1), VEGFR3 (FLT4, CD309), PDGFR-.alpha. (CD140a), PDGFR-.beta. (CD140b) Endoglin, CLEC14, Tem1-8, and Tie2. Further examples may include A33, CAMPATH-1 (CDw52), Carcinoembryonic antigen (CEA), Carboanhydrase IX (MN/CA IX), CD21, CD25, CD30, CD34, CD37, CD44v6, CD45, CD133, de2-7 EGFR, EGFRvIII, EpCAM, Ep-CAM, Folate-binding protein, G250, Fms-like tyrosine kinase 3 (FLT-3, CD135), c-Kit (CD117), CSFIR (CD115), HLA-DR, IGFR, IL-2 receptor, IL3R, MCSP (Melanoma-associated cell surface chondroitin sulphate proteoglycane), Muc-1, Prostate-specific membrane antigen (PSMA), Prostate stem cell antigen (PSCA), Prostate specific antigen (PSA), and TAG-72. Examples of antigens expressed on the extracellular matrix of tumors are tenascin and the fibroblast activating protein (FAP).

In the context of the present invention, “tumor antigen” or “hyperproliferative disorder antigen” or “antigen associated with a hyperproliferative disorder,” refers to antigens that are common to specific hyperproliferative disorders such as cancer. The antigens discussed herein are merely included by way of example. The list is not intended to be exclusive and further examples will be readily apparent to those of skill in the art.

Tumor antigens are proteins that are produced by tumor cells that elicit an immune response, particularly T-cell mediated immune responses. The selection of the antigen binding moiety of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigen are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

### PD-(L)1 Axis Inhibitors

In some embodiments, the invention provides a combination of an anti-Siglec-9 antibody of the invention and a PD-(L)1 axis inhibitor. In various embodiments, the composition comprises an inhibitor of one or more gene or protein in the PD-(L)1 axis. In various embodiments, the present invention includes compositions and methods of decreasing the level or activity of one or more gene or protein in the PD-(L)1 axis. It will be understood by one skilled in the art, based upon the disclosure provided herein, that a decrease in the level or activity of one or more gene or protein in the PD-(L)1 axis encompasses the decrease in the expression of the biomarker, including transcription, translation, or both. The skilled artisan will also appreciate, once armed with the teachings of the present invention, that a decrease in the level or activity of one or more gene or protein in the PD-(L)1 axis includes a decrease in the amount of polypeptide, a decrease in the amount of mRNA, a decrease in transcription, a decrease in translation, or a combination thereof; and it also includes decreasing any activity of one or more gene or protein in the PD-(L)1 axis as well.

Exemplary inhibitors of the PD-(L)1 axis include, but are not limited to, a small interfering RNA (siRNA), a microRNA, an antisense nucleic acid, a ribozyme, an expression vector encoding a transdominant negative mutant, an antibody, a fragment of an antibody, a fusion protein, an aptamer, a peptide and a small molecule.

One skilled in the art will appreciate, based on the disclosure provided herein, that one way to decrease the mRNA and/or protein levels of one or more PD-(L)1 axis protein in a cell is by reducing or inhibiting expression of the nucleic acid encoding the PD-(L)1 axis protein. Thus, the protein level of a PD-(L)1 axis protein in a cell can be decreased using a molecule or compound that inhibits or reduces gene expression such as, for example, siRNA, an antisense molecule or a ribozyme. However, the invention should not be limited to these examples.

In one embodiment, RNAi is used to decrease the level or activity of a PD-(L)1 axis protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. Chemical modification to siRNAs can aid in intravenous systemic delivery. Optimizing siRNAs involves consideration of overall G/C content, C/T content at the termini, Tm and the nucleotide content of the 3′ overhang. Therefore, the present invention also includes methods of decreasing levels of one or more PD-(L)1 axis protein using RNAi technology.

In some embodiments, the invention includes an isolated nucleic acid encoding an inhibitor, such as a protein, an antibody, an siRNA or an antisense molecule operably linked to a nucleic acid comprising a promoter/regulatory sequence such that the nucleic acid is preferably capable of directing expression of the inhibitor encoded by the nucleic acid. Thus, the invention encompasses expression vectors and methods for the introduction of exogenous DNA into cells with concomitant expression of the exogenous DNA in the cells.

In order to assess the expression of the inhibitor, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin

When the inhibitor of the invention is a small molecule, a small molecule antagonist may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

In another aspect of the invention, one or more protein in the PD-(L)1 axis can be inhibited by way of inactivating and/or sequestering the protein(s). As such, inhibiting the effects of one or more protein in the PD-(L)1 axis can be accomplished by using a transdominant negative mutant.

In one embodiment, an antibody specific for one or more protein in the PD-(L)1 axis may be used. As will be understood by one skilled in the art, any antibody that can recognize and bind to an antigen of interest is useful in the present invention. Methods of making and using antibodies are well known in the art. For example, polyclonal antibodies useful in the present invention are generated by immunizing rabbits according to standard immunological techniques well-known in the art. Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

Exemplary anti-PD-(L)1 axis antibodies include, but are not limited to, nivolumab (OPDIVO®), pembrolimumab (KEYTRUDA®), sintilimab, cemiplimab (LIBTAYO®), tripolibamab, tislelizumab, spartalizumab, camrelizumab, dostralimab, genolimzumab or cetrelimab, or antibodies that bind PD-L1, such as PD-L1 antibodies are envafolimab, atezolizumab (TECENTRIQ®), durvalumab (IMFINZI®), avelumab (BAVENCIO®), REGN2810, pidilizumab, MEDI0680, PDR001, PF-06801591, BGB-A317, TSR-042, and SHR-1210.

### Method of Delivery of the Composition

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal, intranasal, intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

### Treatment Methods

In one embodiment, the invention provides a method for treatment or prevention of a disease or disorder which would benefit from an increase in NK cell function or activity. Exemplary diseases and disorders that can be treated using the compositions and methods of the invention include, but are not limited to cancer and infectious diseases.

The following are non-limiting examples of cancers that can be diagnosed or treated by the disclosed methods and compositions: acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

In one embodiment, the compositions are used to treat cancers having a high level of sialic acid. Cancers associated with high levels of sialic acid include, but are not limited to, ovarian cancer, melanoma, renal cell carcinoma, prostate cancer, colon cancer, breast cancer, head and neck squamous cell carcinoma, skin cancer and oral cancer.

### Bacterial Infections

In one embodiment, the infectious disease or disorder is associated with a bacterium. In some embodiments, the bacterium can be from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and

### Verrucomicrobia.

The bacterium can be a gram-positive bacterium or a gram-negative bacterium. The bacterium can be an aerobic bacterium or an anerobic bacterium. The bacterium can be an autotrophic bacterium or a heterotrophic bacterium. The bacterium can be a mesophile, a neutrophile, an extremophile, an acidophile, an alkaliphile, a thermophile, a psychrophile, a halophile, or an osmophile.

The bacterium can be an anthrax bacterium, an antibiotic resistant bacterium, a disease-causing bacterium, a food poisoning bacterium, an infectious bacterium, Salmonella bacterium, Staphylococcus bacterium, Streptococcus bacterium, or tetanus bacterium. The bacterium can be a mycobacteria, Clostridium tetani, Yersinia pestis, Bacillus anthracis, methicillin-resistant Staphylococcus aureus (MRSA), or Clostridium difficile.

### Viral Infections

In one embodiment, the infectious disease or disorder is associated with a bacterium. In some embodiments, the virus is from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. The viral antigen can be from human immunodeficiency virus (HIV), Chikungunya virus (CHIKV), dengue fever virus, papilloma viruses, for example, human papilloma virus (HPV), polio virus, hepatitis viruses, for example, hepatitis A virus (HAV), hepatitis B virus (HBV), hepatitis C virus (HCV), hepatitis D virus (HDV), and hepatitis E virus (HEV), smallpox virus (Variola major and minor), vaccinia virus, influenza virus, rhinoviruses, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, lassa virus, arenavirus, severe acute respiratory syndrome-related coronavirus (SARS), Middle East respiratory syndrome-related coronavirus (MERS), severe acute respiratory syndrome-related coronavirus 2 (SARS COV 2) or a cancer causing virus.

### Parasitic Infections

In one embodiment, the infectious disease or disorder is associated with a parasite. In some embodiments, the parasite can be a protozoa, helminth, or ectoparasite. The helminth (i.e., worm) can be a flatworm (e.g., flukes and tapeworms), a thorny-headed worm, or a round worm (e.g., pinworms). The ectoparasite can be lice, fleas, ticks, and mites.

The parasite can be any parasite causing any one of the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

The parasite can be Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus-lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

### Fungal Infection

In one embodiment, the infectious disease or disorder is associated with a fungus. In some embodiments, the fungus can be Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

### Cancer Therapy

In one embodiment, the invention provides methods of treating or preventing cancer, or of treating and preventing growth or metastasis of tumors. Related aspects, illustrated of the invention provide methods of preventing, aiding in the prevention, and/or reducing metastasis of hyperplastic or tumor cells in an individual.

In one embodiment, the compositions are used to treat cancers having a high level of sialic acid, including, but not limited to, ovarian cancer, melanoma, renal cell carcinoma, prostate cancer, colon cancer, breast cancer, head and neck squamous cell carcinoma, and oral cancer.

One aspect of the invention provides a method of inhibiting metastasis in an individual in need thereof, the method comprising administering to the individual an effective amount of a nucleic acid molecule encoding a multivalent antibody of the invention, wherein the multivalent antibody is specific for the cancer to be treated. The invention further provides a method of inhibiting metastasis in an individual in need thereof, the method comprising administering to the individual an effective metastasis-inhibiting amount of a nucleic acid molecule encoding a multivalent antibody of the invention, wherein the multivalent antibody is specific for the cancer to be treated.

In some embodiments of treating or preventing cancer, or of treating and preventing metastasis of tumors in an individual in need thereof, a second agent is administered to the individual, such as an antineoplastic agent. In some embodiments, the second agent comprises a second metastasis-inhibiting agent, such as a plasminogen antagonist, or an adenosine deaminase antagonist. In other embodiments, the second agent is an angiogenesis inhibiting agent.

The compositions of the invention can be used to prevent, abate, minimize, control, and/or lessen cancer in humans and animals. The compositions of the invention can also be used to slow the rate of primary tumor growth. The compositions of the invention when administered to a subject in need of treatment can be used to stop the spread of cancer cells. As such, an effective amount of a nucleic acid molecule encoding a multivalent antibody of the invention, wherein the multivalent antibody is specific for the cancer to be treated can be administered as part of a combination therapy with one or more drugs or other pharmaceutical agents. When used as part of the combination therapy, the decrease in metastasis and reduction in primary tumor growth afforded by the compositions of the invention allows for a more effective and efficient use of any pharmaceutical or drug therapy being used to treat the patient. In addition, control of metastasis by the compositions of the invention affords the subject a greater ability to concentrate the disease in one location.

In one embodiment, the invention provides a method to treat cancer metastasis comprising treating the subject prior to, concurrently with, or subsequently to the treatment with a composition of the invention, with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The compounds of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the invention include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the compositions of the invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

### Use in Combination

In some embodiments, the present invention provides a method of treating, protecting against, and/or preventing cancer, or a disease or disorder associated with cancer in a subject in need thereof by administering a combination of composition comprising a synthetic Siglec-9 antibody, a fragment thereof, a variant thereof or nucleic acid molecule encoding the same and a composition comprising a tumor antigen, a fragment thereof, a variant thereof or a nucleic acid molecule encoding the same.

In one embodiment, the composition comprising the synthetic anti-Siglec-9 antibody, a fragment thereof, a variant thereof or nucleic acid molecule encoding the same and the composition comprising a tumor antigen, a fragment thereof, a variant thereof or a nucleic acid molecule encoding the same may be administered using any suitable method such that a combination of the anti-Siglec-9 and tumor antigen are both present in the subject. In one embodiment, the method may comprise administration of a first composition comprising the synthetic Siglec-9 antibody, a fragment thereof, a variant thereof or nucleic acid molecule encoding the same of the invention by any of the methods described in detail elsewhere herein and administration of a second composition comprising a tumor antigen, a fragment thereof, a variant thereof or a nucleic acid molecule encoding the same antigen less than 1, less than 2, less than 3, less than 4, less than 5, less than 6, less than 7, less than 8, less than 9 or less than 10 days following administration of the synthetic Siglec-9 antibody, a fragment thereof, a variant thereof or nucleic acid molecule encoding the same. In one embodiment, the method may comprise administration of a first composition comprising the synthetic Siglec-9 antibody, a fragment thereof, a variant thereof or nucleic acid molecule encoding the same of the invention by any of the methods described in detail above and administration of a second composition comprising a tumor antigen, a fragment thereof, a variant thereof or a nucleic acid molecule encoding the same more than 1, more than 2, more than 3, more than 4, more than 5, more than 6, more than 7, more than 8, more than 9 or more than 10 days following administration of the synthetic anti-Siglec-9 antibody, a fragment thereof, a variant thereof or nucleic acid molecule encoding the same. In one embodiment, the method may comprise administration of a first composition comprising a tumor antigen, a fragment thereof, a variant thereof or a nucleic acid molecule encoding the same and administration of a second composition comprising the synthetic anti-Siglec-9 antibody, a fragment thereof, a variant thereof or nucleic acid molecule encoding the same of the invention by any of the methods described in detail above less than 1, less than 2, less than 3, less than 4, less than 5, less than 6, less than 7, less than 8, less than 9 or less than 10 days following administration of the composition comprising a tumor antigen, a fragment thereof, a variant thereof or a nucleic acid molecule encoding the same. In one embodiment, the method may comprise administration of a first composition comprising a tumor antigen, a fragment thereof, a variant thereof or a nucleic acid molecule encoding the same and administration of a second composition comprising the synthetic anti-Siglec-9 antibody, a fragment thereof, a variant thereof or nucleic acid molecule encoding the same of the invention by any of the methods described in detail above more than 1, more than 2, more than 3, more than 4, more than 5, more than 6, more than 7, more than 8, more than 9 or more than 10 days following administration of a composition comprising a tumor antigen, a fragment thereof, a variant thereof or a nucleic acid molecule encoding the same. In one embodiment, the method may comprise administration of a first composition comprising the synthetic anti-Siglec-9 antibody, a fragment thereof, a variant thereof or nucleic acid molecule encoding the same of the invention by any of the methods described in detail above and a second composition comprising a tumor antigen, a fragment thereof, a variant thereof or a nucleic acid molecule encoding the same concurrently. In one embodiment, the method may comprise administration of a single composition comprising the synthetic anti-Siglec-9 antibody, a fragment thereof, a variant thereof or nucleic acid molecule encoding the same of the invention and a tumor antigen, a fragment thereof, a variant thereof or a nucleic acid molecule encoding the same.

The present invention is further illustrated in the following Examples. It should be understood that these Examples, while indicating exemplary embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Generation of Siglec-9 Antibodies Using H2L2 Mice

Sialic acid-binding receptors, such as Siglecs and Selectins, are well-positioned to be exploited by cancer hyper-sialylation. Sialic acids (Sias) are monosaccharides typically found at the outermost ends of complex glycan chains that decorate all vertebrate cell surfaces. Sias are essential for embryonic development and mediate important intrinsic organismal functions. 70 human genes are known to be involved in Sias biology, more than 10 have been documented to be human-specific. Most of these human-specific genetic changes are in Sia-recognizing Ig-like lectin (SIGLEC) genes, found on most types of leukocytes, to the immunological synapse. Up-regulation of sialosides has long been correlated with poor prognosis and decreased immunogenicity of tumors.

Siglec-9 is expressed on monocytes, macrophages, natural killer (NK) cells, dendritic cells, neutrophils, and a subset of T cells. Siglec-9 is an inhibitory receptor whose signals 1) inhibit NK and T cell anti-tumor function, and 2) modulate the immune functions of myeloid cells.

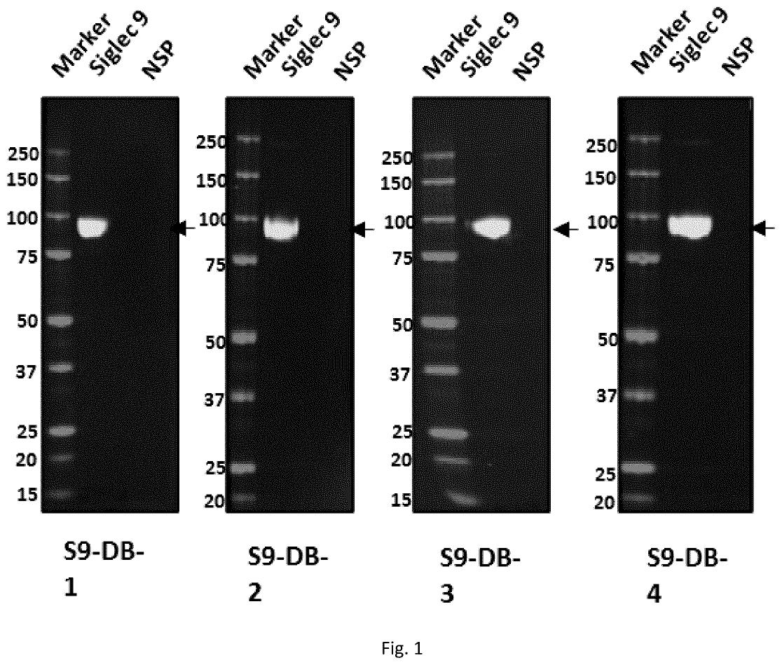

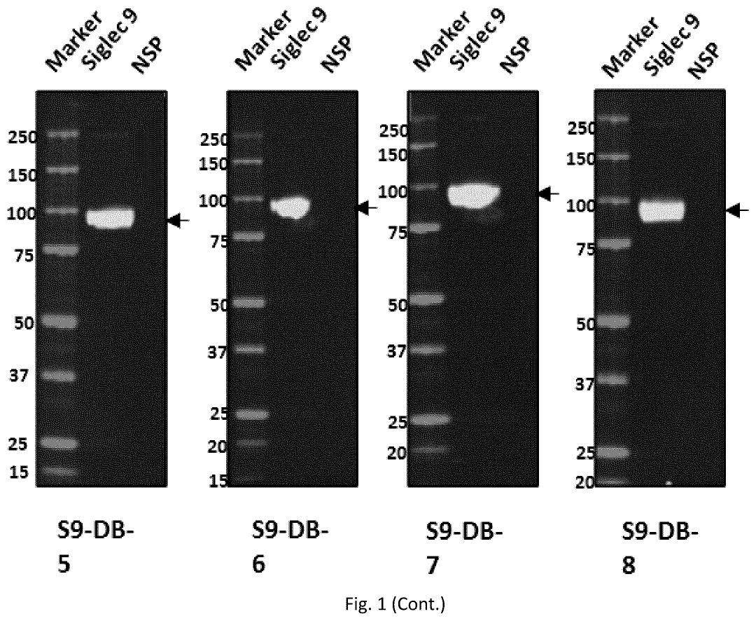

The experiments described herein demonstrate the development and characterization of a Siglec-9 antibodies (FIG. 1). In these experiments, hybridoma supernatants demonstrated specificity and binding to human Siglec-9.

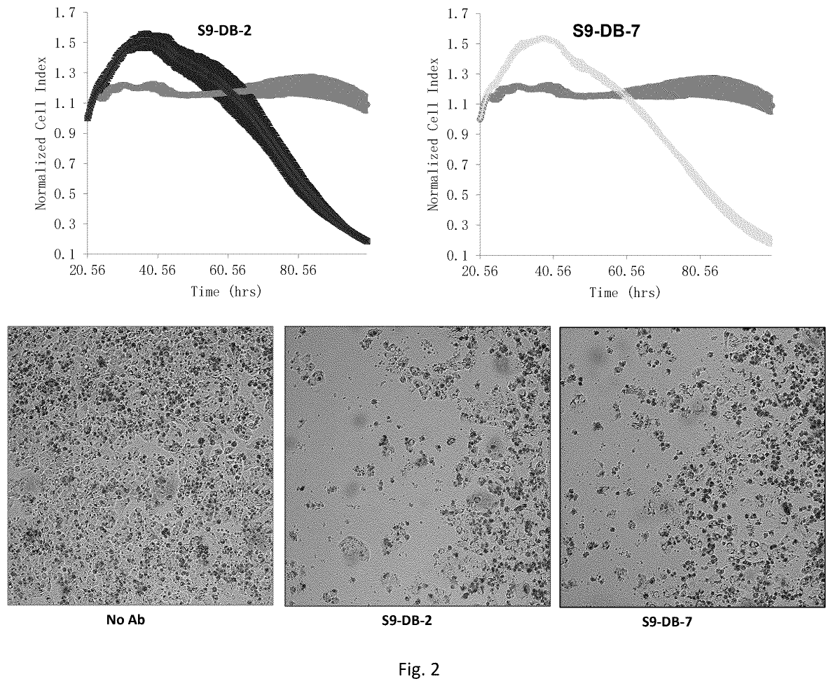

It was observed that the antibodies exhibited cytotoxicity to OVISE cells (FIG. 2). OVISE cells are ovarian clear cell adenocarcinoma derived from metastatic site: Pelvis; innominate bone of a 40-year-old Japanese female. Experiments were designed to test activity of the antibodies. Target cells: OVISE (20,000 cells/well); Effector cells: Human PBMCs; E (Effector): T (Target)-5:1; Effector cells and antibodies (1:10) were added at time point 20 and 56 hrs. Images were captured at 100 hrs whereby it was observed that the Siglec 9 antibodies drove killing in OVISE cells.

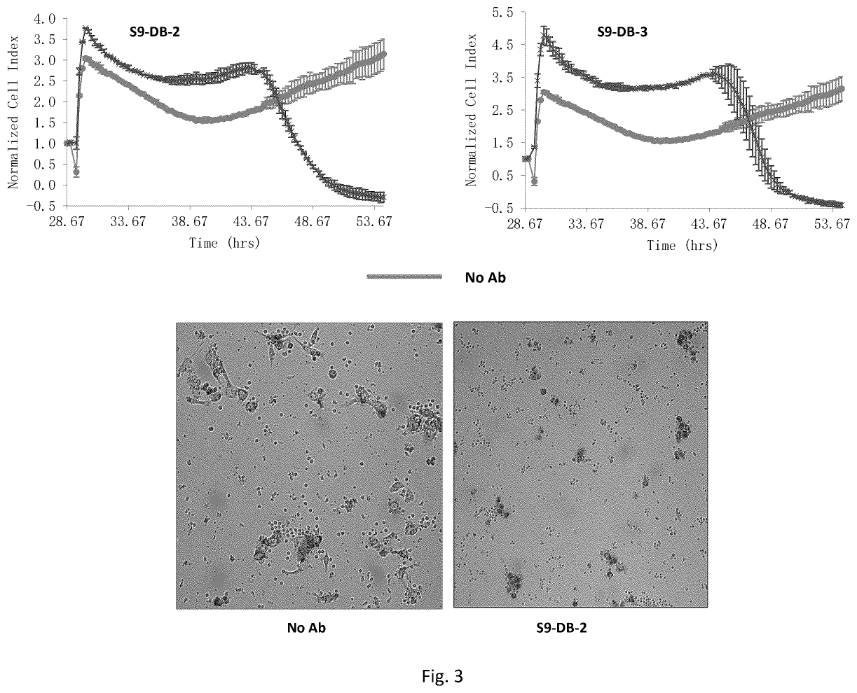

It was also observed that the antibodies exhibited cytotoxicity to OVCAR10 cells (FIG. 3). OVCAR10 is an ovarian human carcinoma cell line. Experiments were designed to test activity of the antibodies. Target cells: OVCAR10 (10,000 cells/well); Effector cells: Human PBMCs E: T=5:1; Effector cells and antibodies were added at 28.67 hrs. Images were captured at 53 hr whereby it was observed that the Siglec 9 antibodies drive killing in OVCAR10 cells. See FIG. 3.

The antibodies did not show cytotoxicity in TC-1 cells which was used as a control target. TC-1 is a mouse cell line where Siglec 9 is not present on the cells.

### Example 2: Sequences-CDR Sequences Underlined

HB9-4G4 (S9-DB-2)
Heavy chain
(SEQ ID NO: 4)
EVQLVESGGGLVKPGGSLRLSCAASGFTFSNAWMSWVRQAPGKGLEWVGR
IKSKTDGGTTDYAAPVKGRFTISRDDSKNTLYLQMNSLKTEDTAVYYCTT
GWELQDYYYYGMDVWGQGTTVTVSS
HB9-4G4 (S9-DB-2)
Heavy chain
(SEQ ID NO: 8)
GAGGTGCAGCTGGTGGAGTCGGGGGGAGGCTTGGTAAAGCCTGGGGGGTC
CCTTAGACTCTCCTGTGCAGCCTCTGGATTCACTTTCAGTAACGCCTGGA
TGAGCTGGGTCCGCCAGGCTCCAGGGAAGGGGCTGGAGTGGGTTGGCCGT
ATTAAAAGCAAAACTGATGGTGGGACAACAGACTACGCTGCACCCGTGAA
AGGCAGATTCACCATCTCAAGAGATGATTCAAAAAACACGCTGTATCTGC
AAATGAACAGCCTGAAAACCGAGGACACAGCCGTGTATTACTGTACCACA
GGGTGGGAGCTACAGGACTACTACTACTACGGTATGGACGTCTGGGGCCA
AGGGACCACGGTCACCGTCTCCTCA
HB9-4G4 (S9-DB-2)
Light chain
(SEQ ID NO: 12)
IVMTQSPATLSVSPGERATLSCRASQSVSSNLAWYQQKPGQAPRLLIYGA
STRATGIPARFSGSGSGTEFTLTISSLQSEDFAVYYCQQYNNWPLTFGGG
TKVEIK
HB9-4G4 (S9-DB-2)
Light chain
(SEQ ID NO: 16)
ATAGTGATGACGCAGTCTCCAGCCACCCTGTCTGTGTCTCCAGGGGAAAG
AGCCACCCTCTCCTGCAGGGCCAGTCAGAGTGTTAGCAGCAACTTAGCCT
GGTACCAGCAGAAACCTGGCCAGGCTCCCAGGCTCCTCATCTATGGTGCA
TCCACCAGGGCCACTGGTATCCCAGCCAGGTTCAGTGGCAGTGGGTCTGG
GACAGAGTTCACTCTCACCATCAGCAGCCTGCAGTCTGAAGATTTTGCAG
TTTATTACTGTCAGCAGTATAATAACTGGCCGCTCACTTTCGGCGGAGGG
ACCAAGGTGGAGATCAAA
HB9-6A8 (S9-DB-3)
Heavy chain
(SEQ ID NO: 20)
QVQLQQWGAGLLKPSETLSLTCAVYGGSFSGYYWSWIRQPPGKGLEWIGE
INHSGSTNYNPSLKSRVTISVDTSKNQFSLKLSSVTAADTAVYYCARGDC
SGGSCPYWYFDLWGRGTLVTVSS
HB9-6A8 (S9-DB-3)
Heavy chain
(SEQ ID NO: 24)
CAGGTGCAGCTACAGCAGTGGGGCGCAGGACTGTTGAAGCCTTCGGAGAC
CCTGTCCCTCACCTGCGCTGTCTATGGTGGGTCCTTCAGTGGTTACTACT
GGAGCTGGATCCGCCAGCCCCCAGGGAAGGGGCTGGAGTGGATTGGGGAA
ATCAATCATAGTGGAAGCACCAACTACAACCCGTCCCTCAAGAGTCGAGT
CACCATATCAGTAGACACGTCCAAGAACCAGTTCTCCCTGAAGCTGAGCT
CTGTGACCGCCGCGGACACGGCTGTGTATTACTGTGCGAGAGGTGATTGT
AGTGGTGGTAGCTGTCCTTACTGGTACTTCGATCTCTGGGGCCGTGGCAC
CCTGGTCACTGTCTCCTCA
HB9-6A8 (S9-DB-3)
Light chain
(SEQ ID NO: 28)
DIVMTQSPLSLPVTPGEPASISCRSSQSLLHSNGYNYLDWYLQKPGQSPQ
LLIYLGSNRASGVPDRFSGSGSGTDFTLKISRVEAEDVGVYYCMQALQTP
RTFGQGTKVEIK
HB9-6A8 (S9-DB-3)
Light chain
(SEQ ID NO: 32)
GATATTGTGATGACTCAGTCTCCACTCTCCCTGCCCGTCACCCCTGGAGA
GCCGGCCTCCATCTCCTGCAGGTCTAGTCAGAGCCTCCTGCATAGTAATG
GATACAACTATTTGGATTGGTACCTGCAGAAGCCAGGGCAGTCTCCACAG
CTCCTGATCTATTTGGGTTCTAATCGGGCCTCCGGGGTCCCTGACAGGTT
CAGTGGCAGTGGATCAGGCACAGATTTTACACTGAAAATCAGCAGAGTGG
AGGCTGAGGATGTTGGGGTTTATTACTGCATGCAAGCTCTACAAACTCCT
CGGACGTTCGGCCAAGGGACCAAGGTGGAAATCAAA
HB9-6F3 (S9-DB-4)
Heavy chain
(SEQ ID NO: 36)
EVQLLESGGGLVQPGGSLRLSCEASGFTFRNYAMSWVRQAPGKGLEWVSA
IRGSGSRTYYADSVKGRFTISRDNSKNTLYLQMNSLRAEDTAVYYCAKDE
GFGDLLAHYVMDAWGQGASVTVSS
HB9-6F3 (S9-DB-4)
Heavy chain
(SEQ ID NO: 40)
GAGGTGCAGCTGTTGGAGTCTGGGGGAGGCTTGGTACAGCCTGGGGGGTC
CCTGAGACTCTCCTGTGAAGCCTCAGGATTCACCTTTAGAAACTATGCCA
TGAGCTGGGTCCGCCAGGCTCCAGGGAAGGGGCTGGAGTGGGTCTCAGCT
ATTCGTGGTAGTGGTAGTAGAACATACTACGCAGACTCTGTGAAGGGCCG
GTTCACCATCTCCAGAGACAATTCCAAGAACACGCTGTATCTGCAGATGA
ACAGCCTGAGAGCCGAGGACACGGCCGTGTATTACTGCGCGAAAGATGAG
GGGTTCGGGGACTTATTAGCGCACTATGTTATGGATGCCTGGGGTCAAGG
AGCTTCAGTCACTGTCTCCTCA
HB9-6F3 (S9-DB-4)
Light chain
(SEQ ID NO: 44)
EIVMTQSPATLSVSPGERATLSCRASQSVSSNLAWYQQKPGQAPRLLIYG
ASTRATGIPARFSGSGSGTEFTLTISSLQSEDFAVYYCQQYNNWPITFGQ
GTRLEIK
HB9-6F3 (S9-DB-4)
Light chain
(SEQ ID NO: 48)
GAAATAGTGATGACGCAGTCTCCAGCCACCCTGTCTGTGTCTCCAGGGGA
AAGAGCCACCCTCTCCTGCAGGGCCAGTCAGAGTGTTAGCAGCAACTTAG
CCTGGTACCAGCAGAAACCTGGCCAGGCTCCCAGGCTCCTCATCTATGGT
GCATCCACCAGGGCCACTGGTATCCCAGCCAGGTTCAGTGGCAGTGGGTC
TGGGACAGAGTTCACTCTCACCATCAGCAGCCTGCAGTCTGAAGATTTTG
CAGTTTATTACTGTCAGCAGTATAATAACTGGCCGATCACCTTCGGCCAA
GGGACACGACTGGAGATTAAA
HB9-9B11 (S9-DB-5)
Heavy chain
(SEQ ID NO: 52)
EVQLLESGGGLVQPGGSLRLSCAASGFTFSNYAMNWVRQAPGKGLEWVSA
ISMSGGSTYYADSVKGRFTISRDNSKNTLYLQMNTLRAEDTAVYYCAKDE
YSSGWYQFDYWGQGTLVTVSS
HB9-9B11 (S9-DB-5)
Heavy chain
(SEQ ID NO: 56)
GAGGTGCAGTTGTTGGAGTCTGGGGGGGGCTTGGTACAGCCTGGGGGGTC
CCTGAGACTCTCCTGTGCAGCCTCTGGATTCACCTTTAGCAACTATGCCA
TGAACTGGGTCCGCCAGGCTCCAGGGAAGGGGCTGGAGTGGGTCTCAGCT
ATTAGTATGAGTGGTGGTAGCACATACTATGCAGACTCCGTGAAGGGCCG
GTTCACCATCTCCAGAGACAATTCCAAGAACACGCTGTATCTGCAAATGA
ACACCCTGAGAGCCGAGGACACGGCCGTATATTACTGTGCGAAAGACGAA
TATAGCAGTGGCTGGTACCAATTTGACTATTGGGGCCAGGGAACCCTGGT
CACCGTCTCCTCA
HB9-9B11 (S9-DB-5)
Light chain
(SEQ ID NO: 60)
DIQMTQSPSSLSASVGDRVTITCRASQSISSYLNWYQQKPGKAPKLLIYA
ASSLQSGVPSRFSGSGSGTDFTLTISSLOPEDFATYYCQQSYSTPLTFGG
GTKVEIK
HB9-9B11 (S9-DB-5)
Light chain
(SEQ ID NO: 64)
GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCT
GCATCCAGTTTGCAAAGTGGGGTCCCATCAAGGTTCAGTGGCAGTGGATC
TGGGACAGATTTCACTCTCACCATCAGCAGTCTGCAACCTGAAGATTTTG
CAACTTACTACTGTCAACAGAGTTACAGTACCCCGCTCACTTTCGGCGGA
GGGACCAAGGTGGAGATCAAA
HB9-10B6 (S9-DB-6)
Heavy chain
(SEQ ID NO: 68)
QVQLVESGGGVVQPGRSLRLSCAASGFTFSTYGMHWVRQAPGKGLEWVAV
TWYDEYNKYYADSVKGRFTISRDNSKNMLYLQMNSLRAEDTAMYYCARNV
LRYFDWTLDYWGQGTLVTVSS
HB9-10B6 (S9-DB-6)
Heavy chain
(SEQ ID NO: 72)
CAGGTGCAGCTGGTGGAGTCTGGGGGAGGCGTGGTCCAGCCTGGGAGGTC
CCTGAGACTCTCCTGTGCAGCGTCTGGATTCACCTTCAGTACCTATGGCA
TGCACTGGGTCCGCCAGGCTCCAGGCAAGGGGCTGGAGTGGGTGGCAGTT
ACATGGTATGATGAATATAATAAATACTATGCAGACTCCGTGAAGGGCCG
CTTCACCATCTCCAGAGACAATTCCAAGAACATGTTGTATCTGCAAATGA
ACAGCCTGAGAGCCGAGGACACGGCTATGTATTACTGTGCGAGGAACGTA
TTACGATATTTTGACTGGACCCTTGACTACTGGGGCCAGGGAACCCTGGT
CACCGTCTCCTCG
HB9-10B6 (S9-DB-6)
Light chain
(SEQ ID NO: 76)
IVLTQSPATLSLSPGERATLSCRASQSVSSYLAWYQQKPGQAPRLLIYDA
SNRATGIPARFSGSGSGTDFTLTISSLEPEDFAVYYCQQRSNWPPTFGQG
TKVEIK
HB9-10B6 (S9-DB-6)
Light chain
(SEQ ID NO: 80)
ATTGTGTTGACACAGTCTCCAGCCACCCTGTCTTTGTCTCCAGGGGAAAG
AGCCACCCTCTCCTGCAGGGCCAGTCAGAGTGTTAGCAGCTACTTAGCCT
GGTATCAACAGAAACCTGGCCAGGCTCCCAGGCTCCTCATCTATGATGCA
TCCAACAGGGCCACTGGCATCCCAGCCAGGTTCAGTGGCAGTGGGTCTGG
GACAGACTTCACTCTCACCATCAGCAGCCTAGAGCCTGAAGATTTTGCAG
TTTATTACTGTCAGCAGCGTAGCAACTGGCCTCCCACTTTTGGCCAGGGG
ACCAAGGTGGAGATCAAA
HB9-10E3 (S9-DB-7)
Heavy chain
(SEQ ID NO: 84)
EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKAG
IAVAGGWYFDLWGRGTLVTVSS
HB9-10E3 (S9-DB-7)
Heavy chain
(SEQ ID NO: 88)
GAGGTGCAGCTGTTGGAGTCTGGGGGAGGCTTGGTACAGCCTGGGGGGTC
CCTGAGACTCTCCTGTGCAGCCTCTGGATTCACCTTTAGCAGCTATGCCA
TGAGCTGGGTCCGCCAGGCTCCAGGGAAGGGGCTGGAGTGGGTCTCAGCT
ATTAGTGGTAGTGGTGGTAGCACATACTACGCAGACTCCGTGAAGGGCCG
GTTCACCATCTCCAGAGACAATTCCAAGAACACGCTGTATCTGCAAATGA
ACAGCCTGAGAGCCGAGGACACGGCCGTATATTACTGTGCGAAAGCCGGT
ATAGCAGTGGCTGGGGGATGGTACTTCGATCTCTGGGGCCGTGGCACCCT
GGTCACTGTCTCCTCA
HB9-10E3 (S9-DB-7)
Light chain
(SEQ ID NO: 92)
IVLTQSPATLSLSPGERATLSCRASQSVSSYLAWYQQKPGQAPRLLIYDA
SNRATGIPARFSGSGSGTDFTLTISSLEPEDFAVYYCQQRSNWPPTFGQG
TKLEIK
HB9-10E3 (S9-DB-7)
Light chain
(SEQ ID NO: 96)
ATTGTGTTGACACAGTCTCCAGCCACCCTGTCTTTGTCTCCAGGGGAAAG
AGCCACCCTCTCCTGCAGGGCCAGTCAGAGTGTTAGCAGCTACTTAGCCT
GGTATCAACAGAAACCTGGCCAGGCTCCCAGGCTCCTCATCTATGATGCA
TCCACAGGGCCACTGGCATCCCAGCCAGGTTCAGTGGCAGTGGGTCTGGG
ACAGACTTCACTCTCACCATCAGCAGCCTAGAGCCTGAAGATTTTGCAGT
TTATTACTGTCAGCAGCGTAGCAACTGGCCTCCCACTTTTGGCCAGGGGA
CCAAGCTGGAGATCAAA
HB9-11B10 (S9-DB-8)
Heavy Chain
(SEQ ID NO: 100)
EVQLLESGGGLVQPGGSLRLSCAASGFTFSNYAMNWVRQAPGKGLEWVSA
ISMSGGSTYYADSVKGRFTISRDNSKNTLYLQMNTLRAEDTAVYYCAKDE
YSSGWYQFDYWGQGTLVTVSS
HB9-11B10 (S9-DB-8)
Heavy Chain
(SEQ ID NO: 104)
GAGGTGCAGTTGTTGGAGTCTGGGGGGGGCTTGGTACAGCCTGGGGGGTC
CCTGAGACTCTCCTGTGCAGCCTCTGGATTCACCTTTAGCAACTATGCCA
TGAACTGGGTCCGCCAGGCTCCAGGGAAGGGGCTGGAGTGGGTCTCAGCT
ATTAGTATGAGTGGTGGTAGCACATACTATGCAGACTCCGTGAAGGGCCG
GTTCACCATCTCCAGAGACAATTCCAAGAACACGCTGTATCTGCAAATGA
ACACCCTGAGAGCCGAGGACACGGCCGTATATTACTGTGCGAAAGACGAA
TATAGCAGTGGCTGGTACCAATTTGACTATTGGGGCCAGGGAACCCTGGT
CACCGTCTCCTCA
HB9-11B10 (S9-DB-8)
Light chain
(SEQ ID NO: 108)
EIVLTQSPATLSLSPGERATLSCRASQSVNSYLAWYQQKPGQAPRLLIYD
ASIRATGIPARFSGSGSGTDFTLTISSLEPEDFAFYYCQQRSNWPPTFGQ
GTKVEIK
HB9-11B10 (S9-DB-8)
Light chain
(SEQ ID NO: 112)
GAAATTGTGTTGACACAGTCTCCAGCCACCCTGTCTTTGTCTCCAGGGGA
AAGAGCCACCCTCTCCTGCAGGGCCAGTCAGAGTGTTAACAGCTACTTAG
CCTGGTACCAACAGAAACCTGGCCAGGCTCCCAGGCTCCTCATCTATGAT
GCATCCATCAGGGCCACTGGCATCCCAGCCAGGTTCAGTGGCAGTGGGTC
TGGGACAGACTTCACTCTCACCATCAGCAGCCTAGAGCCTGAAGATTTTG
CATTTTATTACTGTCAACAGCGTAGTAACTGGCCTCCGACGTTCGGCCAA
GGGACCAAGGTGGAAATCAAA
HB9-1F11
Heavy Chain:
(SEQ ID NO: 116)
QVQLQQPGAELVKPGASVKLSCKASGYTFTSYWMHWVKQRPGRGLEWIGR
IDPNSGGTKYNEKFKSKATLTVDKPSSTAYMOLSSLTSEDSAVYYCARYD
YYGSSYFDYWGQGTTVTVSS
HB9-1F11
(SEQ ID NO: 120)
CAAGTGCAGCTGCAGCAGCCTGGCGCCGAGCTGGTGAAACCTGGAGCCTC
CGTGAAGCTGAGCTGTAAGGCCAGCGGCTACACCTTCACCAGCTACTGGA
TGCACTGGGTCAAGCAGCGGCCAGGAAGAGGCCTGGAATGGATCGGCAGA
ATCGACCCCAACAGCGGCGGCACAAAGTACAACGAGAAGTTCAAGAGCAA
GGCTACACTGACAGTGGACAAGCCTAGCTCCACCGCCTACATGCAGCTGT
CTAGCCTGACCAGCGAAGATAGCGCCGTGTACTACTGCGCCAGATACGAC
TACTACGGCAGCTCTTATTTTGATTACTGGGGCCAGGGCACCACCGTGAC
CGTGTCCAGC
HB9-1F11
Light chain:
(SEQ ID NO: 124)
QAVVTQESALTTSPGETVTLTCRSSTGAVTTSNYANWVQEKPDHLFTGLI
GGTNNRAPGVPARFSGSLIGDKAALTITGAQTEDEAIYFCALWYSNHWVF
GGGTKLTVL
HB9-1F11
(SEQ ID NO: 128)
CAAGCCGTGGTGACACAGGAGAGCGCCCTGACCACATCCCCCGGCGAGAC
AGTGACCCTGACCTGTAGAAGCAGCACAGGCGCTGTGACCACCTCTAACT
ACGCCAACTGGGTCCAGGAAAAACCTGACCACCTGTTTACCGGCCTGATC
GGCGGAACCAACAACCGGGCCCCTGGAGTGCCAGCCAGATTCAGCGGCTC
TCTGATCGGCGACAAGGCCGCTCTCACCATCACCGGCGCCCAGACAGAAG
ATGAGGCCATCTACTTCTGCGCCCTGTGGTACAGCAATCACTGGGTGTTC
GGCGGAGGCACCAAGCTGACAGTGCTG
HB9-4C10
Heavy Chain:
(SEQ ID NO: 132)
EVQLQQSGPELVKPGASVKISCKASGYTFTDYYMNWVKQSHGKSLEWIGG
INPNNGGTSYNQKFKGKATLTVDKSSSTAYMELRSLTSEDSAVYYCARND
GYRGYAMDYWGQGTSVTVSS
HB9-4C10
(SEQ ID NO: 136)
GAGGTGCAGCTGCAGCAATCTGGACCTGAGCTGGTGAAGCCTGGCGCTTC
TGTGAAGATCTCTTGTAAAGCCAGCGGCTACACCTTCACCGATTACTACA
TGAACTGGGTCAAGCAGAGCCACGGCAAAAGCCTGGAATGGATCGGCGGC
ATCAACCCCAACAACGGCGGAACAAGCTACAACCAGAAGTTCAAGGGCAA
GGCTACCCTGACCGTGGACAAGAGCAGCTCCACCGCCTACATGGAACTGC
GGAGCCTGACAAGCGAGGACAGCGCCGTGTACTACTGCGCCAGAAATGAT
GGCTATAGAGGCTACGCCATGGACTACTGGGGACAGGGCACCAGCGTGAC
AGTGTCCTCC
HB9-4C10
Light chain:
(SEQ ID NO: 140)
DIVMSQSPSSLAVSVGEKVTMSCKSSQSLLYSSNQKNYLAWYQQKPGQSP
KLLIYWASTRESGVPDRFTGSGSGTDFTLTISSVKAEDLAVYCCQQYYSN
PPLTFGAGTKLELK
HB9-4C10
(SEQ ID NO: 144)
GACATCGTGATGAGCCAGTCTCCATCTAGCCTGGCCGTGTCCGTGGGCGA
GAAGGTGACCATGTCTTGTAAAAGCTCCCAGAGCCTGCTGTACAGCAGCA
ACCAGAAAAACTACCTGGCCTGGTATCAGCAAAAGCCCGGCCAGAGCCCC
AAGCTCCTGATCTACTGGGCCTCCACACGGGAAAGCGGAGTGCCTGATAG
ATTCACCGGCTCTGGCAGCGGAACCGACTTTACCCTGACCATCAGCAGCG
TCAAGGCCGAGGACCTGGCTGTGTACTGCTGCCAGCAGTACTACAGCAAT
CCTCCTCTGACATTCGGCGCCGGCACAAAGCTGGAACTGAAG
HB9-5B7
Heavy Chain:
(SEQ ID NO: 148)
EVOLVESGGGLVQPGRSLRLSCAASGFTFDDYAMHWVRQAPGKGLEWVSA
ITWNSGHIDYADSVEGRFTISRDNAKNSLYLQMNSLRAEDTAVYYCAKVS
YLSTASSLDYWGQGTLVTVSS
HB9-5B7
(SEQ ID NO: 152)
GAGGTGCAGCTGGTGGAAAGCGGAGGAGGCCTGGTTCAACCCGGCCGGAG
CCTGAGACTGAGCTGTGCCGCTTCTGGCTTTACATTCGACGACTACGCCA
TGCACTGGGTCAGACAGGCCCCTGGCAAGGGCCTCGAGTGGGTGTCCGCC
ATCACCTGGAATAGCGGCCACATCGACTATGCTGATAGCGTGGAAGGCAG
ATTCACCATCAGCAGAGATAACGCCAAGAACTCCCTGTACCTGCAGATGA
ACAGCCTGCGGGCCGAGGACACCGCCGTGTACTACTGCGCCAAAGTGTCC
TACCTGTCTACAGCCAGCAGCCTGGACTACTGGGGCCAGGGCACCCTGGT
GACCGTGTCTAGC
HB9-5B7
Light chain:
(SEQ ID NO: 156)
DIQMTQSPSSLSASVGDRVTITCRASQGISNYLAWYQQKPGQVPKLLIYA
ASALQSGVPSRFSGSGSGTDFTLTITSLQPEDVATYYCHKYNSAPWTFGQ
GTEVEIK
HB9-5B7
(SEQ ID NO: 160)
GACATCCAGATGACCCAGTCCCCTAGCTCTCTGAGCGCCAGCGTGGGCGA
CCGGGTGACCATCACCTGTAGAGCCTCTCAGGGCATCAGCAACTACCTGG
CCTGGTATCAGCAGAAACCCGGACAGGTGCCAAAGCTGCTGATCTACGCC
GCTTCTGCTCTGCAGAGCGGAGTCCCCAGCAGATTCAGCGGCTCCGGCAG
CGGCACAGATTTTACCCTGACCATTACAAGCCTGCAACCTGAGGACGTGG
CCACATACTACTGCCACAAGTACAACAGCGCCCCTTGGACCTTCGGCCAG
GGCACCGAGGTGGAAATCAAG
HB9-7B8
Heavy chain:
(SEQ ID NO: 164)
ELQLLESGGGLVQPGGSLRLSCAASGFTFTNYAMNWVRQAPGKGLEWVSA
ISGSGGRTYYADSVKGRFTISRDNSRNTLFLQMNSLRPEDTAVYYCAKDQ
TSGTTGYPYFAYWGQGTLVTVSS
HB9-7B8
(SEQ ID NO: 168)
GAGCTGCAGCTGCTGGAGTCTGGCGGCGGCCTGGTGCAGCCCGGCGGCTC
CCTGAGGCTGAGCTGCGCCGCCTCCGGCTTTACATTCACAAATTACGCCA
TGAATTGGGTGCGGCAGGCCCCAGGCAAGGGCCTGGAGTGGGTGTCCGCC
ATCTCTGGCTCCGGCGGCAGAACCTATTACGCCGATTCCGTGAAGGGCAG
ATTCACCATCAGCAGAGACAACTCTCGCAATACCCTGTTTCTGCAGATGA
ATAGCCTGAGGCCCGAGGATACAGCCGTGTACTACTGCGCCAAGGATCAG
ACATCTGGCACAACAGGCTACCCTTATTTCGCCTACTGGGGCCAGGGCAC
CCTGGTGACCGTGTCTTCC
HB9-7B8
Light chain:
(SEQ ID NO: 172)
ETVMTQSPATLSVSPGERAILSCRASQSVSSNLVWYQQKPGQAPRLFIYG
ASTRATGIPARFSGSGSGTEFTLTISSLQSEDFAVYYCQQYNNWPRTFGQ
GTKVEIK
HB9-7B8
(SEQ ID NO: 176)
GAGACAGTGATGACCCAGAGCCCAGCCACCCTGTCCGTGTCTCCAGGCGA
GAGGGCCATCCTGTCCTGTAGGGCCTCCcAGAGCGTGTCTTCCAATCTGG
TGTGGTACCAGCAGAAGCCAGGCCAGGCCCCTAGGCTGTTTATCTACGGC
GCCTCCACAAGGGCCACCGGCATCCCTGCCAGATTCTCTGGCTCTGGCTC
CGGCACAGAGTTCACCCTGACAATCTCTTCCCTGCAGTCTGAGGACTTTG
CCGTGTACTATTGCCAGCAGTATAATAACTGGCCTAGGACCTTTGGCCAG
GGCACAAAGGTGGAGATCAAGCA
HB9-14D4
Heavy chain:
(SEQ ID NO: 180)
QVQLVESGGGVVQPGRSLRLSCAASGFAFSSYGMHWVRQAPGKGLEWVAV
IWFDGTKKYYTDSVKGRFTISRDNSKNTLYLQMNTLRAEDTAVYYCARDR
GIGARRGPYYMDVWGKGTTVTVSS
HB9-14D4
(SEQ ID NO: 184)
CAAGTGCAGCTGGTTGAGAGCGGCGGAGGCGTGGTGCAGCCCGGCAGAAG
CCTGAGACTGTCTTGTGCTGCTTCTGGATTTGCCTTCAGCAGCTACGGCA
TGCACTGGGTGCGGCAGGCCCCTGGCAAGGGCCTGGAATGGGTCGCCGTG
ATCTGGTTCGACGGCACAAAGAAGTACTATACAGACAGCGTGAAGGGCAG
ATTCACCATCAGCAGAGATAATAGCAAGAACACCCTGTACCTGCAGATGA
ACACCCTGCGGGCCGAGGACACCGCCGTGTACTACTGCGCCAGAGATAGA
GGCATCGGCGCCAGGCGGGGACCTTACTACATGGACGTGTGGGGCAAAGG
CACCACAGTGACCGTGTCCTCC
HB9-14D4
Light chain:
(SEQ ID NO: 188)
DIQMTQSPSSLTASVGDRVTITCRASQSISSYVNWYQQKPGKAPKVLIFA
ASSLQSGVPSRFSGSGSGTDFTLTISSLQAEDVAVYYCQQYYSTPLTFGG
GTKVEIK
HB9-14D4
(SEQ ID NO: 192)
GATATCCAGATGACACAGAGCCCCAGCAGCCTGACCGCCTCTGTGGGCGA
CCGGGTGACCATCACCTGTAGAGCCAGCCAGTCCATCAGCTCCTACGTGA
ACTGGTATCAGCAGAAACCTGGCAAGGCCCCTAAGGTGCTGATCTTTGCT
GCCAGCAGCCTGCAGAGCGGAGTCCCATCTAGATTCAGCGGCTCTGGCAG
CGGCACCGACTTCACCCTGACAATTAGCTCTCTGCAGGCCGAGGACGTGG
CCGTGTACTACTGCCAGCAATACTACAGCACCCCTCTGACCTTCGGCGGC
GGAACAAAGGTGGAAATCAAG
HB9-14H12
Heavy chain:
(SEQ ID NO: 196)
QVQLKESGPGLVAPSQSLSITCTVSGFLLISNGVHWVRQPPGKGLEWLGV
IWAGGNTNYNSALMSRVSISKDNSKSQVFLKMKSLQTDDTAMYYCARDFY
DYDVFYYAMDYWGQGTSVTVSS
HB9-14H12
(SEQ ID NO: 200)
CAAGTCCAGCTGAAGGAAAGCGGCCCAGGCCTGGTGGCCCCTAGCCAGAG
CCTGAGCATCACATGTACCGTGTCCGGCTTCCTGCTGATCAGCAACGGCG
TGCACTGGGTGCGGCAGCCTCCCGGCAAGGGCCTCGAGTGGCTGGGAGTG
ATCTGGGCTGGCGGAAATACCAACTACAACAGCGCCCTGATGTCTAGAGT
GTCTATCTCTAAGGACAACAGCAAGTCCCAGGTGTTCCTGAAGATGAAAA
GCCTGCAGACCGACGACACCGCCATGTACTACTGCGCCAGAGATTTCTAC
GATTATGACGTTTTTTACTACGCCATGGACTACTGGGGCCAGGGCACCAG
CGTGACAGTGTCCAGC
Light chain:
HB9-14H12
(SEQ ID NO: 204)
QAVVTQESALTTSPGETVPLTCRSSTGTVTTSNFANWVQEKPDHLFTGLI
GGTNNRAPGLPARFSGSLIGDKAALTITGAQTEDEAIFFCALWYSNHFVF
GGGSQLTVL
HB9-14H12
(SEQ ID NO: 208)
CAAGCCGTGGTGACACAGGAATCCGCCCTCACAACCTCTCCAGGCGAGAC
AGTGCCTCTGACCTGTAGAAGCAGCACCGGCACCGTGACCACCAGCAACT
TCGCCAACTGGGTCCAGGAAAAGCCCGACCACCTGTTCACCGGCCTGATC
GGCGGAACAAACAACCGGGCCCCTGGCCTGCCTGCTAGATTCAGCGGCAG
CCTGATCGGAGATAAGGCCGCTCTGACAATCACCGGCGCCCAGACCGAGG
ACGAGGCCATCTTTTTCTGCGCCCTGTGGTACAGCAATCACTTTGTGTTC
GGCGGOGGATCTCAGCTGACCGTGCTG

TABLE 1
SEQUENCES

| | | | | | Full- |
|---|---|---|---|---|---|
| | | | | | length |
| | | CDR1 | CDR2 | CDR3 | Variable |
| | | SEQ | SEQ | SEQ | Region |
| | Sequence | ID | ID | ID | SEQ ID |
| | Type | NO: | NO: | NO: | NO: |
| HB9-4G4 (S9- | Amino acid | 1 | 2 | 3 | 4 |
| DB-2) heavy | Nucleotide | 5 | 6 | 7 | 8 |
| chain | | | | | |
| HB9-4G4 (S9- | Amino acid | 9 | 10 | 11 | 12 |
| DB-2) light | | | GAS | | |
| chain | Nucleotide | 13 | 14 | 15 | 16 |
| | | | GGTGC | | |
| | | | ATCC | | |
| HB9-6A8 (S9- | Amino acid | 17 | 18 | 19 | 20 |
| DB-3) heavy | Nucleotide | 21 | 22 | 23 | 24 |
| chain | | | | | |
| HB9-6A8 (S9- | Amino acid | 25 | 26 | 27 | 28 |
| DB-3) light | | | LGS | | |
| chain | Nucleotide | 29 | 30 | 31 | 32 |
| | | | TTGGG | | |
| | | | TTCT | | |
| HB9-6F3 (S9- | Amino acid | 33 | 34 | 35 | 36 |
| DB-4) heavy | Nucleotide | 37 | 38 | 39 | 40 |
| chain | | | | | |
| HB9-6F3 (S9- | Amino acid | 41 | 42 | 43 | 44 |
| DB-4) light | | | GAS | | |
| chain | Nucleotide | 45 | 46 | 47 | 48 |
| | | | GGTGC | | |
| | | | ATCC | | |
| HB9-9B11 (S9- | Amino acid | 49 | 50 | 51 | 52 |
| DB-5) heavy | Nucleotide | 53 | 54 | 55 | 56 |
| chain | | | | | |
| HB9-9B11 (S9- | Amino acid | 57 | 58 | 59 | 60 |
| DB-5) light | | | AAS | | |
| chain | Nucleotide | 61 | 62 | 63 | 64 |
| | | | GCTGC | | |
| | | | ATCC | | |
| HB9-10B6 (S9- | Amino acid | 65 | 66 | 67 | 68 |
| DB-6) heavy | Nucleotide | 69 | 70 | 71 | 72 |
| chain | | | | | |
| HB9-10B6 (S9- | Amino acid | 73 | 74 | 75 | 76 |
| DB-6) light | | | DAS | | |
| chain | Nucleotide | 77 | 78 | 79 | 80 |
| | | | GATGC | | |
| | | | ATCC | | |
| HB9-10E3 (S9- | Amino acid | 81 | 82 | 83 | 84 |
| DB-7) heavy | Nucleotide | 85 | 86 | 87 | 88 |
| chain | | | | | |
| HB9-10E3 (S9- | Amino acid | 89 | 90 | 91 | 92 |
| DB-7) light | | | DAS | | |
| chain | Nucleotide | 93 | 94 | 95 | 96 |
| | | | GATGC | | |
| | | | ATCC | | |
| HB9-11B10 (S9- | Amino acid | 97 | 98 | 99 | 100 |
| DB-8) heavy | Nucleotide | 101 | 102 | 103 | 104 |
| chain | | | | | |
| HB9-11B10 (S9- | Amino acid | 105 | 106 | 107 | 108 |
| DB-8) light | | | DAS | | |
| chain | Nucleotide | 109 | 110 | 111 | 112 |
| | | | GATGC | | |
| | | | ATCC | | |
| HB9-1F11 heavy | Amino acid | 113 | 114 | 115 | 116 |
| chain | Nucleotide | 117 | 118 | 119 | 120 |
| HB9-1F11 light | Amino acid | 121 | 122 | 123 | 124 |
| chain | | | GTN | | |
| | Nucleotide | 125 | 126 | 127 | 128 |
| | | | GGAAC | | |
| | | | CAAC | | |
| HB9-4C10 heavy | Amino acid | 129 | 130 | 131 | 132 |
| chain | Nucleotide | 133 | 134 | 135 | 136 |
| HB9-4C10 light | Amino acid | 137 | 138 | 139 | 140 |
| chain | | | WAS | | |
| | Nucleotide | 141 | 142 | 143 | 144 |
| | | | TGGGC | | |
| | | | CTCC | | |
| HB9-5B7 heavy | Amino acid | 145 | 146 | 147 | 148 |
| chain | Nucleotide | 149 | 150 | 151 | 152 |
| HB9-5B7 light | Amino acid | 153 | 154 | 155 | 156 |
| chain | | | AAS | | |
| | Nucleotide | 157 | 158 | 159 | 160 |
| | | | GCCGC | | |
| | | | TTCT | | |
| HB9-7B8 heavy | Amino acid | 161 | 162 | 163 | 164 |
| chain | Nucleotide | 165 | 166 | 167 | 168 |
| HB9-7B8 light | Amino acid | 169 | 170 | 171 | 172 |
| chain | | | GAS | | |
| | Nucleotide | 173 | 174 | 175 | 176 |
| | | | GGCGC | | |
| | | | CTCC | | |
| HB9-14D4 heavy | Amino acid | 177 | 178 | 179 | 180 |
| chain | Nucleotide | 181 | 182 | 183 | 184 |
| HB9-14D4 light | Amino acid | 185 | 186 | 187 | 188 |
| chain | | | AAS | | |
| | Nucleotide | 189 | 190 | 191 | 192 |
| | | | GCTGC | | |
| | | | CAGC | | |
| HB9-14H12 | Amino acid | 193 | 194 | 195 | 196 |
| heavy chain | Nucleotide | 197 | 198 | 199 | 200 |
| HB9-14H12 | Amino acid | 201 | 202 | 203 | 204 |
| light chain | | | GTN | | |
| | Nucleotide | 205 | 206 | 207 | 208 |
| | | | GGAAC | | |
| | | | AAAC | | |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A Siglec-9 binding molecule, comprising a Siglec-9 antibody, a Siglec-9 binding antibody fragment or a variant of a Siglec-9 antibody.

2. The Siglec-9 binding molecule of claim 1, wherein the antibody comprises an amino acid sequence selected from the group consisting of:
a) a variable heavy chain sequence comprising the CDR sequences selected from the group consisting of SEQ ID NO: 1-3; SEQ ID NO:17-19; SEQ ID NO: 33-35; SEQ ID NO:49-51; SEQ ID NO: 65-67; SEQ ID NO:81-83; SEQ ID NO:97-99; SEQ ID NO:113-115; SEQ ID NO: 129-131; SEQ ID NO:145-147; SEQ ID NO:161-163; SEQ ID NO:177-179; and SEQ ID NO:193-195;
b) a variable light chain sequence comprising the CDR sequences selected from the group consisting of SEQ ID NO:9-11; SEQ ID NO:25-27; SEQ ID NO: 41-43; SEQ ID NO:57-59; SEQ ID NO: 73-75; SEQ ID NO: 89-91; SEQ ID NO:105-10⁷; SEQ ID NO:121-123; SEQ ID NO:137-139; SEQ ID NO:153-155; SEQ ID NO: 169-171; SEQ ID NO:185-187; and SEQ ID NO:201-203;
c) a sequence having at least 95% identity to a variable heavy chain sequence of one or more of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, and SEQ ID NO: 196;
d) a sequence having at least 95% identity to a variable light chain sequence of one or more of SEQ ID NO: 12, SEQ ID NO: 28, SEQ ID NO: 44, SEQ ID NO: 60, SEQ ID NO: 76, SEQ ID NO: 92, SEQ ID NO: 10⁸, SEQ ID NO: 124, SEQ ID NO: 140, SEQ ID NO: 156, SEQ ID NO: 172, SEQ ID NO: 188, and SEQ ID NO: 204;
e) a fragment comprising at least 80% of the full-length sequence of a variable heavy chain sequence selected from the group consisting of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, and SEQ ID NO: 196; and
f) a fragment comprising at least 80% of the full-length sequence of a variable light chain sequence selected from the group consisting of SEQ ID NO: 12, SEQ ID NO: 28, SEQ ID NO: 44, SEQ ID NO: 60, SEQ ID NO: 76, SEQ ID NO: 92, SEQ ID NO: 10⁸, SEQ ID NO: 124, SEQ ID NO: 140, SEQ ID NO: 156, SEQ ID NO: 172, SEQ ID NO: 188, and SEQ ID NO: 204.

3. The Siglec-9 binding molecule or fragment thereof of claim 1, wherein the Siglec-9 binding molecule comprises an amino acid sequence selected from the group consisting of:
a) a variable heavy chain sequence comprising the CDR sequences of SEQ ID NO: 1-3 and a variable light chain sequence comprising the CDR sequences of SEQ ID NO: 9-11;
b) a variable heavy chain sequence comprising the CDR sequences of SEQ ID NO:17-19 and a variable light chain sequence comprising the CDR sequences of SEQ ID NO:25-27;
c) a variable heavy chain sequence comprising the CDR sequences of SEQ ID NO: 33-35 and a variable light chain sequence comprising the CDR sequences of SEQ ID NO: 41-43;
d) a variable heavy chain sequence comprising the CDR sequences of SEQ ID NO:49-51 and a variable light chain sequence comprising the CDR sequences of SEQ ID NO:57-59;
e) a variable heavy chain sequence comprising the CDR sequences of SEQ ID NO: 65-67 and a variable light chain sequence comprising the CDR sequences of SEQ ID NO: 73-75;
f) a variable heavy chain sequence comprising the CDR sequences of SEQ ID NO: 81-83 and a variable light chain sequence comprising the CDR sequences of SEQ ID NO: 89-91;
g) a variable heavy chain sequence comprising the CDR sequences of SEQ ID NO:97-99 and a variable light chain sequence comprising the CDR sequences of SEQ ID NO:105-10⁷;
h) a variable heavy chain sequence comprising the CDR sequences of SEQ ID NO:113-115 and a variable light chain sequence comprising the CDR sequences of SEQ ID NO:121-123;
i) a variable heavy chain sequence comprising the CDR sequences of SEQ ID NO: 129-131 and a variable light chain sequence comprising the CDR sequences of SEQ ID NO:137-139;
j) a variable heavy chain sequence comprising the CDR sequences of SEQ ID NO: 145-147 and a variable light chain sequence comprising the CDR sequences of SEQ ID NO:153-155;
k) a variable heavy chain sequence comprising the CDR sequences of SEQ ID NO:161-163 and a variable light chain sequence comprising the CDR sequences of SEQ ID NO:169-171;
l) a variable heavy chain sequence comprising the CDR sequences of SEQ ID NO: 177-179 and a variable light chain sequence comprising the CDR sequences of SEQ ID NO:185-187; and
m) a variable heavy chain sequence comprising the CDR sequences of SEQ ID NO:193-195 and a variable light chain sequence comprising the CDR sequences of SEQ ID NO:201-203.

4. The Siglec-9 binding molecule or fragment thereof of claim 1, wherein the Siglec-9 binding molecule comprises an amino acid sequence selected from the group consisting of:
a) a variable heavy chain sequence of SEQ ID NO:4 and a variable light chain sequence of SEQ ID NO:12;
b) a variable heavy chain sequence of SEQ ID NO:20 and a variable light chain sequence of SEQ ID NO:28;
c) a variable heavy chain sequence of SEQ ID NO:36 and a variable light chain sequence of SEQ ID NO:44;
d) a variable heavy chain sequence of SEQ ID NO:52 and a variable light chain sequence of SEQ ID NO:60;
e) a variable heavy chain sequence of SEQ ID NO:68 and a variable light chain sequence of SEQ ID NO:76;
f) a variable heavy chain sequence of SEQ ID NO:84 and a variable light chain sequence of SEQ ID NO:92;
g) a variable heavy chain sequence of SEQ ID NO:100 and a variable light chain sequence of SEQ ID NO:10⁸;
h) a variable heavy chain sequence of SEQ ID NO:116 and a variable light chain sequence of SEQ ID NO:124;
i) a variable heavy chain sequence of SEQ ID NO:132 and a variable light chain sequence of SEQ ID NO:140;
j) a variable heavy chain sequence of SEQ ID NO:148 and a variable light chain sequence of SEQ ID NO:156;
k) a variable heavy chain sequence of SEQ ID NO:164 and a variable light chain sequence of SEQ ID NO:172;
l) a variable heavy chain sequence of SEQ ID NO:180 and a variable light chain sequence of SEQ ID NO:188; and
m) a variable heavy chain sequence of SEQ ID NO:196 and a variable light chain sequence of SEQ ID NO:204.

5. The Siglec-9 binding molecule or fragment thereof of claim 1, wherein the Siglec-9 binding molecule comprises a fusion protein comprising a Siglec-9 antibody, a Siglec-9 binding antibody fragment or a variant of a Siglec-9 antibody.

6. A nucleic acid molecule, or combination of nucleic acid molecules, comprising one or more nucleotide sequence encoding a Siglec-9 binding molecule of any one of claims 1-5.

7. The nucleic acid molecule of claim 6, wherein the nucleic acid molecule comprises one or more nucleotide sequence selected from the group consisting of:
a) a nucleotide sequence encoding a variable heavy chain sequence wherein the nucleotide sequence comprises CDR encoding sequences selected from the group consisting of SEQ ID NO:5-7; SEQ ID NO:21-23; SEQ ID NO:37-39; SEQ ID NO: 53-55; SEQ ID NO:69-71; SEQ ID NO:85-87; SEQ ID NO: 101-103; SEQ ID NO: 117-119; SEQ ID NO:133-135; SEQ ID NO:149-151; SEQ ID NO:165-167; SEQ ID NO: 181-183; and SEQ ID NO:197-199;
b) a nucleotide sequence encoding a variable light chain sequence wherein the nucleotide sequence comprises CDR encoding sequences selected from the group consisting of SEQ ID NO: 13-15; SEQ ID NO:29-31; SEQ ID NO:45-47; SEQ ID NO: 61-63; SEQ ID NO: 77-79; SEQ ID NO:93-95; SEQ ID NO: 10⁹-111; SEQ ID NO: 125-127; SEQ ID NO: 141-143; SEQ ID NO: 157-159; SEQ ID NO: 173-175; SEQ ID NO: 189-191; and SEQ ID NO:205-207;
c) a nucleotide sequence having at least 95% identity to a variable heavy chain encoding sequence selected from the group consisting of SEQ ID NO:8; SEQ ID NO:24; SEQ ID NO:40; SEQ ID NO:56; SEQ ID NO:72; SEQ ID NO:88; SEQ ID NO: 104; SEQ ID NO: 120; SEQ ID NO:136; SEQ ID NO: 152; SEQ ID NO: 168; SEQ ID NO: 184; and SEQ ID NO:200;
d) a nucleotide sequence having at least 95% identity to a variable light chain encoding sequence selected from the group consisting of SEQ ID NO:16; SEQ ID NO:32; SEQ ID NO:48; SEQ ID NO:64; SEQ ID NO:80; SEQ ID NO:96; SEQ ID NO: 112; SEQ ID NO: 128; SEQ ID NO: 144; SEQ ID NO: 160; SEQ ID NO: 176; SEQ ID NO: 192; and SEQ ID NO:208;
e) a fragment comprising at least 80% of the full-length sequence of a variable heavy chain encoding sequence selected from the group consisting of SEQ ID NO: 8; SEQ ID NO:24; SEQ ID NO:40; SEQ ID NO:56; SEQ ID NO:72; SEQ ID NO:88; SEQ ID NO: 104; SEQ ID NO:120; SEQ ID NO:136; SEQ ID NO:152; SEQ ID NO:168; SEQ ID NO:184; and SEQ ID NO:200; and
f) a fragment comprising at least 80% of the full-length sequence of a variable light chain encoding sequence selected from the group consisting of SEQ ID NO: 16; SEQ ID NO:32; SEQ ID NO:48; SEQ ID NO:64; SEQ ID NO:80; SEQ ID NO: 96; SEQ ID NO:112; SEQ ID NO:128; SEQ ID NO:144; SEQ ID NO:160; SEQ ID NO: 176; SEQ ID NO: 192; and SEQ ID NO:208.

8. The nucleic acid molecule(s) of claim 7, wherein the nucleic acid molecule(s) comprises nucleotide sequences selected from the group consisting of:
a) a first nucleotide sequence comprising SEQ ID NO:5-7, encoding a variable heavy chain sequence, and a second nucleotide sequence comprising SEQ ID NO: 13-15 encoding a variable light chain sequence;
b) a first nucleotide sequence comprising SEQ ID NO:21-23, encoding a variable heavy chain sequence, and a second nucleotide sequence comprising SEQ ID NO: 29-31 encoding a variable light chain sequence;
c) a first nucleotide sequence comprising SEQ ID NO:37-39, encoding a variable heavy chain sequence, and a second nucleotide sequence comprising SEQ ID NO: 45-47 encoding a variable light chain sequence;
d) a first nucleotide sequence comprising SEQ ID NO:53-55, encoding a variable heavy chain sequence, and a second nucleotide sequence comprising SEQ ID NO: 61-63 encoding a variable light chain sequence;
e) a first nucleotide sequence comprising SEQ ID NO:69-71, encoding a variable heavy chain sequence, and a second nucleotide sequence comprising SEQ ID NO: 77-79 encoding a variable light chain sequence;
f) a first nucleotide sequence comprising SEQ ID NO:85-87, encoding a variable heavy chain sequence, and a second nucleotide sequence comprising SEQ ID NO: 93-95 encoding a variable light chain sequence;
g) a first nucleotide sequence comprising SEQ ID NO:101-103, encoding a variable heavy chain sequence, and a second nucleotide sequence comprising SEQ ID NO: 10⁹-111 encoding a variable light chain sequence;
h) a first nucleotide sequence comprising SEQ ID NO:117-119, encoding a variable heavy chain sequence, and a second nucleotide sequence comprising SEQ ID NO: 125-127 encoding a variable light chain sequence;
i) a first nucleotide sequence comprising SEQ ID NO:133-135, encoding a variable heavy chain sequence, and a second nucleotide sequence comprising SEQ ID NO: 141-143 encoding a variable light chain sequence;
j) a first nucleotide sequence comprising SEQ ID NO:149-151, encoding a variable heavy chain sequence, and a second nucleotide sequence comprising SEQ ID NO: 157-159 encoding a variable light chain sequence;
k) a first nucleotide sequence comprising SEQ ID NO: 165-167, encoding a variable heavy chain sequence, and a second nucleotide sequence comprising SEQ ID NO: 173-175 encoding a variable light chain sequence;
l) a first nucleotide sequence comprising SEQ ID NO: 181-183, encoding a variable heavy chain sequence, and a second nucleotide sequence comprising SEQ ID NO: 189-191 encoding a variable light chain sequence; and
m) a first nucleotide sequence comprising SEQ ID NO:197-199, encoding a variable heavy chain sequence, and a second nucleotide sequence comprising SEQ ID NO: 205-207 encoding a variable light chain sequence.

9. The nucleic acid molecule(s) of claim 7, wherein the nucleic acid molecule(s) comprises nucleotide sequences selected from the group consisting of:
a) a first nucleotide sequence comprising SEQ ID NO:8, encoding a variable heavy chain sequence, and a second nucleotide sequence comprising SEQ ID NO:16 encoding a variable light chain sequence;
b) a first nucleotide sequence comprising SEQ ID NO:24, encoding a variable heavy chain sequence, and a second nucleotide sequence comprising SEQ ID NO: 32 encoding a variable light chain sequence;
c) a first nucleotide sequence comprising SEQ ID NO:40, encoding a variable heavy chain sequence, and a second nucleotide sequence comprising SEQ ID NO: 48 encoding a variable light chain sequence;
d) a first nucleotide sequence comprising SEQ ID NO:56, encoding a variable heavy chain sequence, and a second nucleotide sequence comprising SEQ ID NO: 64 encoding a variable light chain sequence;
e) a first nucleotide sequence comprising SEQ ID NO:72, encoding a variable heavy chain sequence, and a second nucleotide sequence comprising SEQ ID NO: 80 encoding a variable light chain sequence;
f) a first nucleotide sequence comprising SEQ ID NO:88, encoding a variable heavy chain sequence, and a second nucleotide sequence comprising SEQ ID NO: 96 encoding a variable light chain sequence;
g) a first nucleotide sequence comprising SEQ ID NO: 104, encoding a variable heavy chain sequence, and a second nucleotide sequence comprising SEQ ID NO: 112 encoding a variable light chain sequence;
h) a first nucleotide sequence comprising SEQ ID NO: 120, encoding a variable heavy chain sequence, and a second nucleotide sequence comprising SEQ ID NO: 128 encoding a variable light chain sequence;
i) a first nucleotide sequence comprising SEQ ID NO: 136, encoding a variable heavy chain sequence, and a second nucleotide sequence comprising SEQ ID NO: 144 encoding a variable light chain sequence;
j) a first nucleotide sequence comprising SEQ ID NO: 152, encoding a variable heavy chain sequence, and a second nucleotide sequence comprising SEQ ID NO: 160 encoding a variable light chain sequence;
k) a first nucleotide sequence comprising SEQ ID NO:168, encoding a variable heavy chain sequence, and a second nucleotide sequence comprising SEQ ID NO: 176 encoding a variable light chain sequence;
l) a first nucleotide sequence comprising SEQ ID NO:184, encoding a variable heavy chain sequence, and a second nucleotide sequence comprising SEQ ID NO: 192 encoding a variable light chain sequence; and
m) a first nucleotide sequence comprising SEQ ID NO:200, encoding a variable heavy chain sequence, and a second nucleotide sequence comprising SEQ ID NO: 208 encoding a variable light chain sequence.

10. A composition comprising a Siglec-9 binding molecule of any one of claims 1-5.

11. The composition of claim 10, further comprising at least one selected form the group consisting of a pharmaceutically acceptable excipient and an adjuvant.

12. The composition of claim 10, further comprising a PD-(L)1 axis inhibitor.

13. The composition of claim 10, further comprising a tumor antigen or a nucleic acid molecule encoding a tumor antigen.

14. The composition of claim 10, comprising a delivery vehicle comprising the Siglec-7 binding molecule.

15. The composition of claim 14, wherein the delivery vehicle is a lipid nanoparticle.

16. The composition of any one of claims 10-15, further comprising at least one selected from the group consisting of a pharmaceutically acceptable excipient and an adjuvant.

17. A composition comprising one or more nucleic acid molecule(s) encoding a Siglec-9 binding molecule of any one of claims 6-9.

18. The composition of claim 17, wherein the composition comprises a combination of a first expression vector comprising a nucleotide sequence encoding a heavy chain sequence and a second expression vector comprising a nucleotide sequence encoding a light chain sequence.

19. The composition of claim 17, wherein the composition comprises a single expression vector comprising a first nucleotide sequence encoding a heavy chain sequence and a second nucleotide sequence encoding a light chain sequence.

20. The composition of claim 17, further comprising a PD-(L)1 axis inhibitor.

21. The composition of claim 17, further comprising a tumor antigen or a nucleic acid molecule encoding a tumor antigen.

22. The composition of claim 17, comprising a delivery vehicle comprising the nucleic acid molecule(s).

23. The composition of claim 22, wherein the delivery vehicle is a lipid nanoparticle.

24. The composition of any one of claims 17-23, further comprising at least one selected from the group consisting of a pharmaceutically acceptable excipient and an adjuvant.

25. A method of treating or preventing a disease or disorder in a subject in need thereof, the method comprising administering a Siglec-9 binding molecule of any one of claims 1-5, one or more nucleic acid molecules encoding a Siglec-9 binding molecule of any one of claims 6-9, or a composition of any one of claims 10-24 to the subject.

26. The method of claim 25, wherein the disease or disorder is a cancer, or a disease or disorder associated with cancer.

27. The method of claim 26, wherein the cancer has increased levels of sialic acid.

28. The method of claim 27, wherein the cancer is selected from the group consisting of ovarian cancer, melanoma, renal cell carcinoma, prostate cancer, colon cancer, breast cancer, head and neck squamous cell carcinoma, skin cancer and oral cancer.

29. The method of claim 25, wherein the disease or disorder is an infectious disease or disorder.

30. A method of increasing natural killer cell function in a subject in need thereof, the method comprising administering a Siglec-9 binding molecule of any one of claims 1-5, one or more nucleic acid molecules encoding a Siglec-9 binding molecule of any one of claims 6-9, or a composition of any one of claims 10-24 to the subject.
